(19) United States
(12) Patent Application Publication
(10) Pub. No.: US 2013/0131059 A1
(43) Pub. Date: May 23, 2013

(54) METHOD FOR TREATING OPHTHALMIC DISEASES USING KINASE INHIBITOR COMPOUNDS IN PRODRUG FORMS

(71) Applicant: Inspire Pharmaceuticals, Inc., Whitehouse Station, NJ (US)

(72) Inventors: John W. LAMPE, Dedham, MA (US); Sammy R. SHAVER, Chapel Hill, NC (US); Paul S. WATSON, Carrboro, NC (US)

(73) Assignee: INSPIRE PHARMACEUTICALS, INC., Whitehouse Station, NJ (US)

(21) Appl. No.: 13/744,321

(22) Filed: Jan. 17, 2013

Related U.S. Application Data
(63) Continuation of application No. PCT/US2011/045244, filed on Jul. 25, 2011.
(60) Provisional application No. 61/368,183, filed on Jul. 27, 2010.

(51) Int. Cl. C07D 401/12; C07D 403/12; C07D 401/14

(52) U.S. Cl. CPC C07D 401/12; C07D 401/14; C07D 403/12
USPC 514/234.5; 546/143; 514/310; 548/361.1; 514/406; 546/199; 514/322; 544/130; 544/364; 514/253.09

(57) ABSTRACT

This invention is directed to prodrugs of rho kinase (ROCK) inhibitors. These prodrugs are in general the ester or the amide derivatives of the parent compounds. These prodrugs are often weak inhibitors of ROCK, but their parent compounds have good activities. Upon instillation into the eyes, the ester or the amide group of these prodrugs is rapidly hydrolyzed into alcohol, amine, or acid, and the prodrugs are converted into the active base compounds. The prodrugs of ROCK inhibitors provide several advantages such as delivery of higher concentrations of the active species into the target site and reduction of ocular discomfort. The invention is also directed to a method of treating ophthalmic diseases such as glaucoma, allergic conjunctivitis, macular edema, macular degeneration, and blepharitis, by administering an effective amount of a ROCK prodrug compound of Formula I to the eyes of the patient in need of.

This application is a continuation of PCT/US2011/045244, filed Jul. 25, 2011; which claims the priority of U.S. Provisional Application No. 61/368,183, filed Jul. 27, 2010. The contents of the above-identified applications are incorporated herein by reference in their entireties.

### TECHNICAL FIELD

This invention relates to synthetic rho-associated kinase (ROCK) inhibiting compounds in a prodrug form, and the methods of making such compounds. The invention also relates to methods of using such compounds in preventing or treating diseases or conditions that are affected or can be assisted by altering the integrity or rearrangement of the cytoskeleton, including but not exclusive of actomyosin interactions, tight junctional and focal adhesion complexes. Particularly, this invention relates to methods of treating ophthalmic diseases such as disorders in which intraocular pressure is elevated, for example primary open-angle glaucoma, using such compounds.

### BACKGROUND OF THE INVENTION

### Rho Kinase as a Target

The Rho family of small GTP binding proteins can be activated by several extracellular stimuli such as growth factors, hormones and mechanic stress and function as a molecular signaling switch by cycling between an inactive GDP-bound form and an active GTP-bound form to elicit cellular responses. Rho kinase (ROCK) functions as a key downstream mediator of Rho and exists as two isoforms (ROCK 1 and ROCK 2) that are ubiquitously expressed. ROCKs are serine/threonine kinases that regulate the function of a number of substrates including cytoskeletal proteins such as adducin, moesin, Na⁺—H⁺ exchanger 1 (NHE1), LIM-kinase and vimentin, contractile proteins such as the myosin light chain phosphatase binding subunit (MYPT-1), CPI-17, myosin light chain and calponin, microtubule associated proteins such as Tau and MAP-2, neuronal growth cone associated proteins such as CRMP-2, signaling proteins such as PTEN and transcription factors such as serum response factor (Loirand et al, Circ Res 98:322-334 (2006)). ROCK is also required for cellular transformation induced by RhoA. As a key intermediary of multiple signaling pathways, ROCK regulates a diverse array of cellular phenomena including cytoskeletal rearrangement, actin stress fiber formation, proliferation, chemotaxis, cytokinesis, cytokine and chemokine secretion, endothelial or epithelial cell junction integrity, apoptosis, transcriptional activation and smooth muscle contraction. As a result of these cellular actions, ROCK regulates many physiologic processes such as vasoconstriction, bronchoconstriction, tissue remodeling, inflammation, edema, platelet aggregation and proliferative disorders.

One well documented example of ROCK activity is in smooth muscle contraction. In smooth muscle cells ROCK mediates calcium sensitization and smooth muscle contraction. Agonists (noradrenaline, acetylcholine, endothelin, etc.) that bind to G protein coupled receptors produce contraction by increasing both the cytosolic Ca²⁺ concentration and the Ca²⁺ sensitivity of the contractile apparatus. The Ca²⁺-sensitizing effect of smooth muscle constricting agents is ascribed to ROCK-mediated phosphorylation of MYPT-1, the regulatory subunit of myosin light chain phosphatase (MLCP), which inhibits the activity of MLCP resulting in enhanced phosphorylation of the myosin light chain and smooth muscle contraction (WO 2005/003101A2, WO 2005/034866A2).

ROCK inhibitors have utility in treating many disorders. One example is the treatment of ophthalmic diseases such as but not limited to: glaucoma, allergic conjunctivitis, macular edema and degeneration, and blepharitis. Glaucoma is an ophthalmic disease that leads to irreversible visual impairment. It is the fourth most common cause of blindness and the second most common cause of visual loss in the United States, and the most common cause of irreversible visual loss among African-Americans. Generally speaking, the disease is characterized by a progressive optic neuropathy caused at least in part by deleterious effects resulting from increased intraocular pressure. In normal individuals, intraocular pressures range from 12 to 20 mm Hg, averaging approximately 16 mm Hg. However, in individuals suffering from primary open angle glaucoma, intraocular pressures generally rise above 22 to 30 mm Hg. In angle closure or acute glaucoma intraocular pressure can reach as high as 70 mm Hg leading to blindness within only a few days.

The most common allergic eye disease, allergic conjunctivitis (AC) can be subdivided into acute, seasonal and perennial. All three types result from classic Type I IgE-mediated hypersensitivity (Abelson, M B., et. al. Surv Ophthalmol; 38(S):115, 1993). Allergic conjunctivitis is a relatively benign ocular disease of young adults (average age of onset of 20 years of age) that causes significant suffering and use of healthcare resources, although it does not threaten vision. Ocular allergy is estimated to affect 20 percent of the population on an annual basis, and the incidence is increasing (Abelson, M B et. al., Surv Ophthalmol., 38(S):115, 1993). AC impacts productivity and while there are a variety of agents available for the treatment of AC, numerous patients still lack good control of symptoms and some are tolerating undesired side effects. Surveys have shown 20% of patients with AC are not fully satisfied with their AC medications and almost 50% feel they receive insufficient attention from their physicians (Mahr, et al., Allergy Asthma Proc, 28(4):404-9, 2007).

Macular edema is a condition that occurs when damaged (or newly formed) blood vessels leak fluid onto the macula, a critical part of the retina for visual acuity, causing it to swell and blur vision. Macular edema is a common problem in diabetic retinopathy, where retinal vessel injury causes edema. Edema also occurs in the proliferative phase of diabetic retinopathy, when newly formed vessels leak fluid into either, or both, the macula and/or vitreous. Macular edema is commonly problematic in age-related macular degeneration (wet form) as well, where newly formed capillaries (angiogenesis) leak fluid into the macula. Age related macular degeneration (AMD) is a progressive eye condition affecting as many as 10 million Americans. AMD is the number one cause of vision loss and legal blindness in adults over 60 in the U.S. As the population ages, and the “baby boomers” advance into their 60's and 70's, a virtual epidemic of AMD will be prevalent. The disease affects the macula of the eye, where the sharpest central vision occurs. Although it rarely results in complete blindness, it robs the individual of all but the outermost, peripheral vision, leaving only dim images or black holes at the center of vision.

Blepharitis, also known as Lid Margin Disease (LMD), is a non-contagious inflammation of the eyelids that manifests itself through scaling and flaking around the eyelashes, excess sebum production and oily scaly discharge, mucopurulent discharge, and matted, hard crusts around the lashes. Accumulation of crust, discharge or debris on the eyelashes and lid margins creates an ideal environment for overgrowth of the staphylococcal bacteria naturally found on the skin of the eyelids and increases the chance of infection, allergic reaction and tear break down. Blepharitis disturbs the production of the critical, outer lipid layer of the tear film which causes the entire tear to evaporate, resulting in dry eye. A reduced tear quantity doesn't properly dilute bacteria and irritants, nor wash inflammatory products away from the lashes and lid margin, so they accumulate and lead to further inflammation worsening the cycle of disease, with blepharitis, meibomian gland dysfunction and dry eye perpetuating each other.

U.S. Pat. Nos. 6,586,425, 6,110,912, and 5,798,380 disclose a method for the treatment of glaucoma using compounds that affect the actin filament integrity of the eye to enhance aqueous humor outflow. These patents also specifically disclose kinase inhibitors as well as latrunculin-A, latrunculin-B, swinholide-A, and jasplakinolide, which cause a perturbation of the actin cytoskeleton and tight junctional complexes in the trabecular meshwork or the modulation of its interactions with the underlying membrane. Perturbation of the cytoskeleton and the associated adhesions reduces the resistance of aqueous humor flow through the trabecular meshwork and thereby reduces intraocular pressure.

U.S. Publication No. 20080214614 discloses a method of lowering intraocular pressure by administering to a subject a synthetic cytoskeletal active compound that is an inhibitor of rho-associated protein kinase.

Esterases are present in all anterior segment tissues of the eye. The activity can be microsomal, cytostolic, or extracellular. There are at least two types of esterases, primarily being acetyl cholinesterase and butyryl cholinesterase. Additionally, enzymes such as peptidases and carbonic anhydrase, both found on and within the ocular surface, possess esterase-like activity. As shown by Lee et. al. (Curr. Eye Res., 4:1117-1125, 1985), 1-naphthylacetate was hydrolyzed to the carboxylic acid derivative within the conjunctiva, corneal epithelia, corneal stroma, ciliary body, and aqueous humor of rabbits.

There exists a need for effective and cost-practical cytoskeletal active compounds to treat glaucoma, to modulate wound healing after trabeculectomy, and to treat other diseases or disorders that are affected by the integrity of the actin cytoskeleton. There exists a need for novel cytoskeletal active compounds that can be obtained using practical synthetic procedures.

### SUMMARY OF THE INVENTION

The present invention is directed to a compound of Formula I, or its pharmaceutically acceptable salt, tautomers thereof.

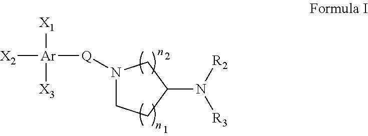

The compounds are prodrugs of rho kinase (ROCK) inhibitors. These prodrugs are in general the ester or the amide derivatives of the parent compounds. Upon instillation into the eyes, the ester or the amide group of these prodrugs is rapidly hydrolyzed into alcohol, amine, or acid, and the prodrugs are converted into the active base compounds.

The invention is also directed to a method of treating ophthalmic diseases such as glaucoma, allergic conjunctivitis, macular edema, macular degeneration, and blepharitis, by administering an effective amount of a ROCK prodrug compound of Formula I to the eyes of a subject in need of.

### BRIEF DESCRIPTION OF THE DRAWINGS

FIG. 1 shows the comparison of ocular tolerability scores between a prodrug (Compound 14) and its parent compound (Compound 49).

FIG. 2 shows the comparison of ocular tolerability scores between prodrugs (Compounds 17-20) and their parent compound (Compound 48). Compound 49 was included in the figure only to show relevance to FIG. 1.

### DETAILED DESCRIPTION OF THE INVENTION

### Definitions

When present, unless otherwise specified, the following terms are generally defined as, but are not limited to, the following:

Halo substituents are taken from fluorine, chlorine, bromine, and iodine.

“Alkyl” refers to groups of from 1 to 12 carbon atoms inclusively, either straight chained or branched, more preferably from 1 to 8 carbon atoms inclusively, and most preferably 1 to 6 carbon atoms inclusively.

“Alkenyl” refers to groups of from 2 to 12 carbon atoms inclusively, either straight or branched containing at least one double bond but optionally containing more than one double bond.

“Alkynyl” refers to groups of from 2 to 12 carbon atoms inclusively, either straight or branched containing at least one triple bond but optionally containing more than one triple bond, and additionally optionally containing one or more double bonded moieties.

“Alkoxy” refers to the group alkyl-O— wherein the alkyl group is as defined above including optionally substituted alkyl groups as also defined above.

“Alkenoxy” refers to the group alkenyl-O— wherein the alkenyl group is as defined above including optionally substituted alkenyl groups as also defined above.

“Alkynoxy” refers to the group alkynyl-O— wherein the alkynyl group is as defined above including optionally substituted alkynyl groups as also defined above.

“Aryl” refers to an unsaturated aromatic carbocyclic group of from 6 to 14 carbon atoms inclusively having a single ring (e.g., phenyl) or multiple condensed rings (e.g., naphthyl or anthryl). Preferred aryls include phenyl, naphthyl and the like.

“Arylalkyl” refers to aryl-alkyl-groups preferably having from 1 to 6 carbon atoms inclusively in the alkyl moiety and from 6 to 10 carbon atoms inclusively in the aryl moiety. Such arylalkyl groups are exemplified by benzyl, phenethyl and the like.

“Arylalkenyl” refers to aryl-alkenyl-groups preferably having from 2 to 6 carbon atoms in the alkenyl moiety and from 6 to 10 carbon atoms inclusively in the aryl moiety.

“Arylalkynyl” refers to aryl-alkynyl-groups preferably having from 2 to 6 carbon atoms inclusively in the alkynyl moiety and from 6 to 10 carbon atoms inclusively in the aryl moiety.

“Cycloalkyl” refers to cyclic alkyl groups of from 3 to 12 carbon atoms inclusively having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantyl, and the like.

“Cycloalkenyl” refers to cyclic alkenyl groups of from 4 to 12 carbon atoms inclusively having a single cyclic ring or multiple condensed rings and at least one point of internal unsaturation, which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Cycloalkylalkyl” refers to cycloalkyl-alkyl-groups preferably having from 1 to 6 carbon atoms inclusively in the alkyl moiety and from 6 to 10 carbon atoms inclusively in the cycloalkyl moiety. Such cycloalkylalkyl groups are exemplified by cyclopropylmethyl, cyclohexylethyl and the like.

“Cycloalkylalkenyl” refers to cycloalkyl-alkenyl-groups preferably having from 2 to 6 carbon atoms inclusively in the alkenyl moiety and from 6 to 10 carbon atoms inclusively in the cycloalkyl moiety. Such cycloalkylalkenyl groups are exemplified by cyclohexylethenyl and the like.

“Cycloalkylalkynyl” refers to cycloalkyl-alkynyl-groups preferably having from 2 to 6 carbon atoms inclusively in the alkynyl moiety and from 6 to 10 carbon atoms inclusively in the cycloalkyl moiety. Such cycloalkylalkynyl groups are exemplified by cyclopropylethynyl and the like.

“Heteroaryl” refers to a monovalent aromatic heterocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to heteroaryl-alkyl-groups preferably having from 1 to 6 carbon atoms inclusively in the alkyl moiety and from 6 to 10 atoms inclusively in the heteroaryl moiety. Such heteroarylalkyl groups are exemplified by pyridylmethyl and the like.

“Heteroarylalkenyl” refers to heteroaryl-alkenyl-groups preferably having from 2 to 6 carbon atoms inclusively in the alkenyl moiety and from 6 to 10 atoms inclusively in the heteroaryl moiety.

“Heteroarylalkynyl” refers to heteroaryl-alkynyl-groups preferably having from 2 to 6 carbon atoms inclusively in the alkynyl moiety and from 6 to 10 atoms inclusively in the heteroaryl moiety.

“Heterocycle” refers to a saturated or unsaturated group having a single ring or multiple condensed rings, from 1 to 8 carbon atoms inclusively and from 1 to 4 hetero atoms inclusively selected from nitrogen, sulfur or oxygen within the ring. Such heterocyclic groups can have a single ring (e.g., piperidinyl, tetrahydrofuryl, morpholinyl, or piperazinyl) or multiple condensed rings (e.g., indolinyl, dihydrobenzofuran or quinuclidinyl). Preferred heterocycles include piperidinyl, pyrrolidinyl and tetrahydrofuryl.

“Heterocycle-alkyl” refers to heterocycle-alkyl-groups preferably having from 1 to 6 carbon atoms inclusively in the alkyl moiety and from 6 to 10 atoms inclusively in the heterocycle moiety. Such heterocycle-alkyl groups are exemplified by morpholino-ethyl, pyrrolidinylmethyl, and the like.

“Heterocycle-alkenyl” refers to heterocycle-alkenyl-groups preferably having from 2 to 6 carbon atoms inclusively in the alkenyl moiety and from 6 to 10 atoms inclusively in the heterocycle moiety.

“Heterocycle-alkynyl” refers to heterocycle-alkynyl-groups preferably having from 2 to 6 carbon atoms inclusively in the alkynyl moiety and from 6 to 10 atoms inclusively in the heterocycle moiety.

Examples of heterocycles and heteroaryls include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

Unless otherwise specified, positions occupied by hydrogen in the foregoing groups can be further substituted with substituents exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

The term “heteroatom-containing substituent” refers to substituents containing at least one non-halogen heteroatom. Examples of such substituents include, but are not limited to, hydroxy, oxo, nitro, methoxy, ethoxy, alkoxy, substituted alkoxy, trifluoromethoxy, haloalkoxy, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, aryloxy, pyridyl, imidazolyl, heteroaryl, substituted heteroaryl, heteroaryloxy, heteroarylalkyl, heteroarylalkenyl, heteroarylalkynyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

“Pharmaceutically acceptable salts” are salts that retain the desired biological activity of the parent compound and do not impart undesired toxicological effects. Pharmaceutically acceptable salt forms include various polymorphs as well as the amorphous form of the different salts derived from acid or base additions. The acid addition salts can be formed with inorganic or organic acids. Illustrative but not restrictive examples of such acids include hydrochloric, hydrobromic, sulfuric, phosphoric, citric, acetic, propionic, benzoic, napthoic, oxalic, succinic, maleic, fumaric, malic, adipic, lactic, tartaric, salicylic, methanesulfonic, 2-hydroxyethanesulfonic, toluenesulfonic, benzenesulfonic, camphorsulfonic, and ethanesulfonic acids. The pharmaceutically acceptable base addition salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C₁₋₄).

A “prodrug” is a precursor of an active drug. A prodrug is converted to an active drug upon administration to a subject.

“Tautomers” are compounds that can exist in one or more forms, called tautomeric forms, which can interconvert by way of a migration of one or more hydrogen atoms in the compound accompanied by a rearrangement in the position of adjacent double bonds. These tautomeric forms are in equilibrium with each other, and the position of this equilibrium will depend on the exact nature of the physical state of the compound. It is understood that where tautomeric forms are possible, the current invention relates to all possible tautomeric forms.

“Solvates” are addition complexes in which a compound of the invention is combined with a pharmaceutically acceptable cosolvent in some fixed proportion. Cosolvents include, but are not limited to, water, methanol, ethanol, 1-propanol, isopropanol, 1-butanol, isobutanol, tert-butanol, acetone, methyl ethyl ketone, acetonitrile, ethyl acetate, benzene, toulene, xylene(s), ethylene glycol, dichloromethane, 1,2-dichloroethane, N-methylformamide, N,N-dimethylformamide, N-methylacetamide, pyridine, dioxane, and diethyl ether. Hydrates are solvates in which the cosolvent is water. It is to be understood that the definitions of compounds of the invention encompass all possible hydrates and solvates, in any proportion, which possess the stated activity.

“An effective amount” is the amount effective to treat a disease by ameliorating the pathological condition or reducing the symptoms of the disease. “An effective amount” is the amount effective to improve at least one of the parameters relevant to measurement of the disease.

The inventors have discovered that certain prodrugs of rho kinase (ROCK) inhibitors are effective as topical ophthalmic agents. These prodrugs are in general the ester or the amide derivatives of the parent compounds (base compounds). These prodrugs contain a metabolically labile, covalent linkage of an ester or amide bond, which is hydrolyzed upon administration to a subject. These prodrugs are often weak inhibitors of ROCK, but their parent compounds have good activities. Upon instillation into the eyes, the ester or the amide group of these prodrugs is rapidly hydrolyzed into alcohol, amine, or acid, and the prodrugs are converted into the active base compounds. The conversion of prodrugs to parent compounds in vivo makes it possible to dose a comparatively weak ROCK inhibitor and achieve a therapeutically useful concentration of an active ROCK inhibitor in the eye. The prodrugs of ROCK inhibitors provide several advantages. The inventors have found through pharmacokinetic studies that these prodrugs, for example, lipophilic esters, are better absorbed into the eye than the corresponding more polar alcohols. This ultimately allows delivery of higher concentrations of the more active species into the target site. The inventors have discovered that when administering a compound in a prodrug form (ester or amide derivatives) rather than the active form (alcohol, amine, or acid) to an eye of an animal, a higher concentration of the active parent compound is present in the aqueous humor. In addition, the prodrugs in some cases reduce levels of undesired effects compared to their more potent parent compounds. For example, some ROCK inhibitor compounds produce an uncomfortable sensation upon installation into the eye. The prodrugs of those ROCK inhibitor compounds may reduce the ocular discomfort that an animal senses.

The prodrug compounds of the present invention are shown in Formula I:

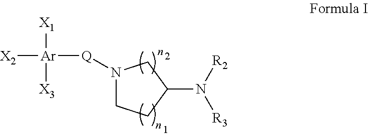

wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by

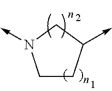

is optionally substituted by alkyl, halo, oxo, OR₆, NR₆R₇, or SR₆;
R₂ is selected from the following heteroaryl systems, optionally substituted:

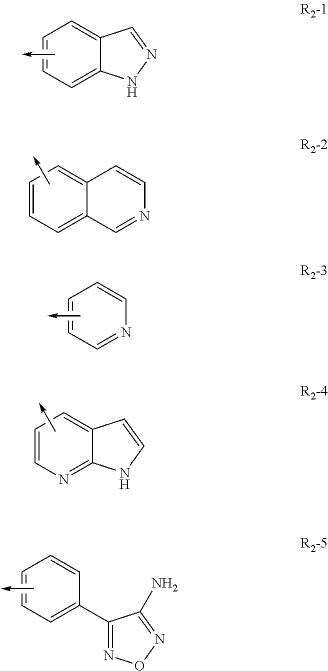

R₃—R₇ are independently H, alkyl, alkenyl, alkynyl, cycloalkyl, cycloalkenyl, cycloalkylalkyl, cycloalkylalkenyl, or cycloalkylalkynyl, optionally substituted;
Ar is a monocyclic or bicyclic aryl or heteroaryl ring, such as phenyl or naphthyl, optionally substituted;
X₁ is -J₁C(O)R₁₀ or -J₁(CR₈R₉)n₄J₂C(O)R₁₀ with n₄=1-6 and J₁ and J₂ are independently O, NR₁₂, or absent;
X₂ and X₃ are independently H, halogen, OR₁₂, NR₁₂R₁₃, SR₁₂, SOR₁₂, SO₂R₁₂, SO₂NR₁₂R₁₃, OCF3, saturated or unsaturated heterocycle, heteroaryl, aryl, alkyl, alkenyl, or alkynyl;
R₈, R₉ are independently H, halogen, alkyl (n=1-3), alkyloxy, alkylthio, or OR₁₁;
R₁₀ is alkyl, alkenyl, heterocycle, aryl, heteroaryl, aralkyl, cycloalkyl, each optionally substituted; or R₁₀ is OR₁₂ or NR₁₂R₁₃;
R₁₁=H or alkyl (n=1-3); and
R₁₂ and R₁₃ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle, optionally substituted.

In Formula I, a preferred Q is (CR₄R₅)ₙ₃, a more preferred Q is CH₂; a preferred n₁ is 1 or 2; a preferred n₂ is 1; a preferred n₃ is 1 or 2; a preferred R₃—R₇ are H; a preferred R₂=R₂−1; a preferred R₂ is R₂−2, a preferred central ring is unsubstituted; a preferred J₂ is O or NR₁₂; a preferred J₁ is absent or O. Preferred Formula I compounds include any combination of the above listed preferred groups.

Formula I represents novel compounds provided that when Q=CH₂; n₁=n₂=1; R₂=R₂−2; R₃=H, Ar=phenyl; X₂ and X₃=H; X₁=OCH₂CH₂OC(O)R₁₂, then R₁₂ is not phenyl.

### Preparation of Compounds of Formula I

General approaches for preparing the compounds of Formula I are described in Scheme 1 and Scheme 2. Those having skill in the art will recognize that the starting materials can be varied and additional steps can be employed to produce compounds encompassed by the present invention. In some cases, protection of certain reactive functionalities may be necessary to achieve some of the above transformations. In general, the need for such protecting groups as well as the conditions necessary to attach and remove such groups will be apparent to those skilled in the art of organic synthesis.

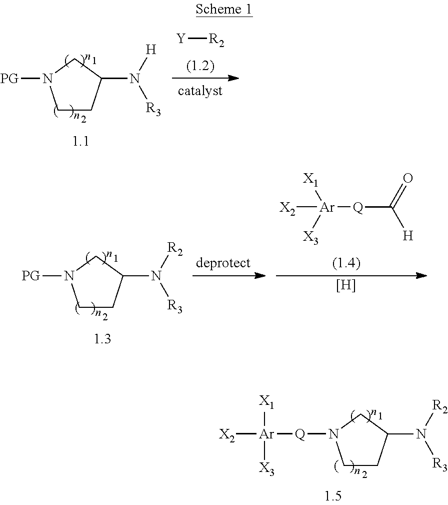

Materials of Formula I using halo-substituted starting materials are prepared by general Scheme 1. For illustration, 5-bromo-isoquinoline (1.2) is reacted with a protected pyrrolidine- or piperidine-amine (1.1, these diamines are readily prepared using preparations well known in the literature) via coupling methods generally involving palladium catalysis to generate intermediate 1.3. The base stable protecting group PG is removed by treatment with an acid (trifluoroacetic acid, for example) and the resulting free amine is coupled with an appropriate aldehyde (1.4) via reductive amination (using a borohydride reagent such as sodium triacetoxyborohydride) to yield the desired product (1.5). As protected diamines are readily available in optically active form using methods well known in the literature, the methods of Scheme 1 provide convenient methods to prepare the compounds of Formula I in optically active form.

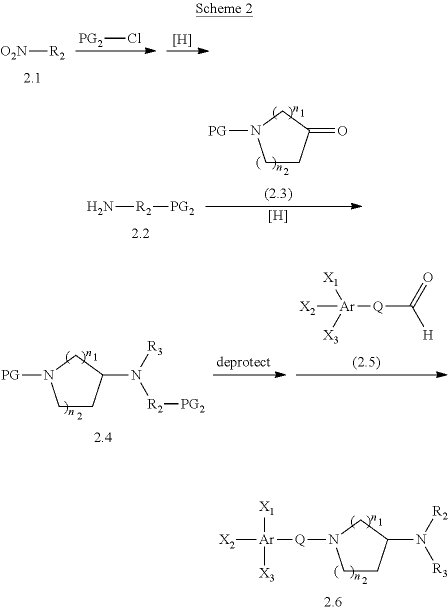

Materials of Formula I using nitro-substituted starting materials are prepared by general Scheme 2. For illustration, 5-nitro-indazole (2.1) is protected with a base resistant protecting group at the 1-position. This protected indazole is subjected to catalytic hydrogenation to generate the 5-amino compound (2.2). Coupling of this compound via reductive amination (using a borohydride reagent such as sodium triacetoxyborohydride) with a suitably chosen protected pyrrolidine or piperidine (2.3, readily prepared using preparations well known in the literature) generates intermediate 2.4. This doubly protected product is fully deprotected with trifluoroacetic acid then coupled with an appropriate aldehyde (2.5, readily prepared using methods well known in the literature) via a second reductive amination using a borohydride reagent (such as sodium triacetoxyborohydride) to yield the desired product (2.6).

The above two synthetic schemes can be modified using well-known procedures, which allow the preparation of other members in the scope of Formula I.

The preparation of specific prodrug compounds 14-46 is illustrated in Examples 14-46.

### Pharmaceutical Composition

The present invention provides pharmaceutical compositions comprising pharmaceutically acceptable formulations comprising a pharmaceutically acceptable carrier and one or more compounds of Formula I, pharmaceutically acceptable salts, solvates, and/or hydrates thereof. The pharmaceutically acceptable carrier can be selected by those skilled in the art using conventional criteria. Pharmaceutically acceptable carriers include, but are not limited to, aqueous- and non-aqueous based solutions, suspensions, emulsions, microemulsions, micellar solutions, gels, and ointments. The pharmaceutically active carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, hydronium, phosphate, citrate, acetate, borate, and tromethamine; antioxidants such as salts, acids and/or bases of bisulfite, sulfite, metabisulfite, thiosulfite, ascorbic acid, acetyl cysteine, cystein, glutathione, butylated hydroxyanisole, butylated hydroxytoluene, tocopherols, and ascorbyl palmitate; surfactants such as phospholipids (e.g., phosphatidylcholine, phosphatidylethanolamine and phosphatidyl inositiol), poloxamers and ploxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and polysaccharides such as dextrans, and glycosaminoglycans such as sodium hyaluronate. Such pharmaceutically acceptable carriers may be preserved against bacterial contamination using well-known preservatives, these include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

In one embodiment of the invention, the compositions are formulated as topical ophthalmic preparations, with a pH of about 3-9, preferably 4 to 8. The compounds of the invention are generally contained in these formulations in an amount of at least 0.001% by weight, for example, 0.001% to 5% by weight, preferably about 0.003% to about 2% by weight, with an amount of about 0.02% to about 1% by weight being most preferred. For topical administration, one to two drops of these formulations are delivered to the surface of the eye one to four times per day according to the routine discretion of a skilled clinician.

In one embodiment of the invention, the compositions are formulated as aqueous pharmaceutical formulations comprising at least one compound of Formula I in an amount of 0.001-2% w/v, and a tonicity agent to maintain a tonicity between 200-400 mOsm/kG, wherein the pH of the formulation is 3-9.

In yet another embodiment, the aqueous pharmaceutical formulation comprises at least one compound of Formula I in an amount of 0.001-2% w/v, one or more complexing and/or solubilizing agents, 0.01-0.5% preservative, 0.01-1% chelating agent, and a tonicity agent to maintain a tonicity between 200-400 mOsm/kG, wherein the pH of the formulation is 4-8. The preferred amount of the compound is 0.01-1% w/v.

The delivery of such ophthalmic preparations may be done using a single unit dose vial wherein the inclusion of a preservative may be precluded. Alternatively, the ophthalmic preparation may be contained in an ophthalmic dropper container intended for multi-use. In such an instance, the multi-use product container may or may not contain a preservative, especially in the event the formulation is self-preserving. Furthermore, the dropper container is designed to deliver a certain fixed volume of product preparation in each drop. The typical drop volume of such an ophthalmic preparation will range from 20-60 μL, preferably 25-55 μL, more preferably 30-50 μL, with 35-50 μL being most preferred.

### Use of the Compounds

Glaucoma is an ophthalmic disease that leads to irreversible visual impairment. Primary open-angle glaucoma is characterized by abnormally high resistance to fluid (aqueous humor) drainage from the eye. Cellular contractility and changes in cell-cell and cell-trabeculae adhesion in the trabecular meshwork are major determinants of the resistance to flow. The compounds of the present invention cause a transient, pharmacological perturbation of both cell contractility and cell adhesions, mainly via disruption of the actomyosin-associated cytoskeletal structures and/or the modulation of their interactions with the membrane. Altering the contractility of trabecular meshwork cells leads to drainage-surface expansion. Loss of cell-cell, cell-trabeculae adhesion may influence paracellular fluid flow across Schlemm's canal or alter the fluid flow pathway through the juxtacanalicular tissue of the trabecular meshwork. Both mechanisms likely reduce the resistance of the trabecular meshwork to fluid flow and thereby reduce intraocular pressure in a therapeutically useful manner.

Regulation of the actin cytoskeleton is important in the modulation of fluid transport. Antimitotic drugs markedly interfere with antidiuretic response, strongly implying that cytoskeleton integrity is essential to this function. This role of the cytoskeleton in controlling the epithelial transport is a necessary step in the translocation of the water channel containing particle aggregates and in their delivery to the apical membrane. Osmolality-dependent reorganization of the cytoskeleton and expression of specific stress proteins are important components of the regulatory systems involved in the adaptation of medullary cells to osmotic stress. The compounds of the present invention are useful in directing epithelial function and modulating fluid transport, particularly modulating fluid transport on the ocular surface.

Rho-associated protein kinase inhibitors, due to their regulation of smooth muscle contractility, are useful in the treatment of vasospasm, specifically retinal vasospasm. Relaxation of retinal vasculature increases perfusion rates thereby providing a neuroprotective mechanism (decreased apoptosis and necrosis) in retinal diseases and retinopathies such as glaucoma, ocular hypertension, age-related macular degeneration or retinitis pigmentosa. Additionally, these kinase inhibitors regulate vascular endothelial permeability and as such can play a vasoprotective role to various atherogenic agents.

The present invention provides a method of reducing intraocular pressure, including treating glaucoma such as primary open-angle glaucoma; a method of treating constriction of the visual field; a method of modulating fluid transport on the ocular surface; a method of controlling vasospasm; a method of increasing tissue perfusion; and a method of vasoprotection to atherogenic agents. The method comprises the steps of identifying a subject in need of treatment, and administering to the subject a compound of Formula I, in an amount effective to alter the actin cytoskeleton, such as by inhibiting actomyosin interactions.

The present invention is also directed to methods of preventing or treating ocular diseases associated with excessive inflammation, proliferation, remodeling, neurite retraction, corneal neurodegeneration, vaso-permeability and edema. Particularly, this invention relates to methods treating ocular diseases such as allergic conjunctivitis, macular edema, macular degeneration, and blepharitis. The method comprises identifying a subject in need of the treatment, and administering to the subject an effective amount of the compound of Formula I to treat the disease.

The method is useful in treating mammals, particularly in treat humans.

In one embodiment, the pharmaceutical composition of the present invention is administered locally to the eye (e.g., topical, intracameral, intravitreal, subretinal, subconjunctival, retrobulbar or via an implant) in the form of ophthalmic formulations. The compounds of the invention can be combined with ophthalmologically acceptable preservatives, surfactants, viscosity enhancers, penetration enhancers, bioadhesives, antioxidants, buffers, sodium chloride, and water to form an aqueous or non-aqueous, sterile ophthalmic suspension, emulsion, microemulsion, gel, or solution to form the compositions of the invention.

The active compounds disclosed herein can be administered to the eyes of a patient by any suitable means, but are preferably administered by administering a liquid or gel suspension of the active compound in the form of drops, spray or gel. Alternatively, the active compounds can be applied to the eye via liposomes. Further, the active compounds can be infused into the tear film via a pump-catheter system. Another embodiment of the present invention involves the active compound contained within a continuous or selective-release device, for example, membranes such as, but not limited to, those employed in the OCUSERT™ System (polymeric ocular inserts for administering drugs). As an additional embodiment, the active compounds can be contained within, carried by, or attached to contact lenses that are placed on the eye. Another embodiment of the present invention involves the active compound contained within a swab or sponge that can be applied to the ocular surface. Another embodiment of the present invention involves the active compound contained within a liquid spray that can be applied to the ocular surface. Another embodiment of the present invention involves an injection of the active compound directly into the lacrimal tissues or onto the eye surface.

The invention is illustrated further by the following examples that are not to be construed as limiting the invention in scope to the specific procedures described in them.

### EXAMPLES

### Example 1

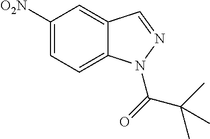 [SMILES: CC(C)(C)C(=O)n1ncc2cc([N+](=O)[O-])ccc21]

### 2,2-Dimethyl-1-(5-nitro-1H-indazol-1-yl)propan-1-one

A 3-neck round bottom flask fitted with a nitrogen inlet and mechanical stirrer was charged with a solution of 5-nitroindazole in tetrahydrofuran. The mixture was cooled to 0° C. and 1.2 equivalents of triethylamine was added. To the mixture was added 1.05 equivalents of pivaloyl chloride dropwise over a period of 15 minutes. The reaction was allowed to warm to 20° C. over a period of 2 hours. The reaction was filtered and concentrated to a dark red oil. To the oil was added methylene chloride and the resulting slurry was stirred vigorously, giving a white precipitate that was isolated by filtration. The solid was dried in a vacuum oven at 40° C. overnight to afford the title compound.

### Example 2

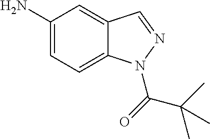 [SMILES: CC(C)(C)C(=O)n1ncc2cc(N)ccc21]

### 1-(5-Amino-1H-indazol-1-yl)-2,2-dimethylpropan-1-one Maleate

Into a 0.5 L stainless steel reaction vessel were added 2,2-dimethyl-1-(5-nitro-1H-indazol-1-yl)propan-1-one (Example 1, 1 equivalent), ethanol and 10% palladium on charcoal (2 mol %). The vessel was sealed, evacuated and refilled with nitrogen three times, and evacuated and refilled with hydrogen to 75 psi. As the hydrogen was consumed, the vessel was refilled until a pressure of 75 psi was maintained. The vessel was degassed and the reaction mixture was removed, filtered over celite, and concentrated to give the desired product as a yellow oil. The crude product was dissolved in ethanol and a solution of maleic acid (1 equivalent) in ethanol was added in one portion. The mixture was stirred vigorously. As a precipitate began to form, the mixture was cooled to 0° C. and stirred for thirty minutes. The precipitate was isolated by filtration and dried in a vacuum oven at 30° C. overnight to provide the title compound as a solid.

### Example 3

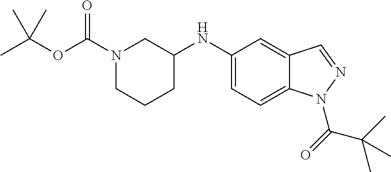 [SMILES: CC(C)(C)OC(=O)N1CCCC(Nc2ccc3c(cnn3C(=O)C(C)(C)C)c2)C1]

### tert-Butyl 3-(1-Pivaloyl-1H-indazol-5-ylamino)piperidine-1-carboxylate

Into a 3-neck round bottom flask fitted with a nitrogen inlet and mechanical stirrer was added tert-butyl 3-oxopiperidine-1-carboxylate and an equimolar amount of 1-(5-amino-1H-indazol-1-yl)-2,2-dimethylpropan-1-one maleate salt (Example 2) in 1,2-dichloroethane. The vessel was purged with nitrogen and stirred at 20° C. for one hour. Sodium triacetoxyborohydride (1.3 equivalents) was added, and the reaction was monitored by analytical TLC to completion. The reaction was quenched with saturated sodium bicarbonate. The organic phase was isolated, dried over MgSO₄, filtered and evaporated to dryness to afford the title compound as a yellow solid.

### Example 4

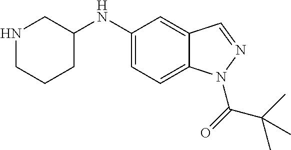 [SMILES: CC(C)(C)C(=O)n1ncc2cc(NC3CCCNC3)ccc21]

### 2,2-Dimethyl-1-(5-(piperidin-3-ylamino)-1H-indazol-1-yl)propan-1-one

Into a 3-neck round bottom flask equipped with an additional funnel and a magnetic stir bar were added tert-butyl 3-(1-pivaloyl-1H-indazol-5-ylamino)piperidine-1-carboxylate (Example 3) and dichloromethane. The mixture was cooled to 0° C. and an excess of trifluoroacetic acid was added dropwise. The reaction was monitored by HPLC for disappearance of the starting material. Upon completion the reaction was concentrated to give the trifluoroacetate salt of the desired product. Residual trifluoroacetic acid was removed under vacuum. The salt was converted to its free base by partitioning between saturated sodium bicarbonate and ethyl acetate. The organic phase was separated, dried over MgSO₄, filtered and concentrated to give the title compound as an amorphous solid.

### Example 5

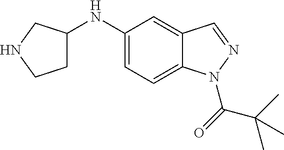 [SMILES: CC(C)(C)C(=O)n1ncc2cc(NC3CCNC3)ccc21]

### 2,2-Dimethyl-1-(5-(pyrrolidin-3-ylamino)-1H-indazol-1-yl)propan-1-one

Reaction of tert-butyl 3-oxopyrrolidine-1-carboxylate and 1-(5-amino-1H-indazol-1-yl)-2,2-dimethylpropan-1-one maleate salt using the method of Example 3 followed by deprotection using the method of Example 4 afforded the title compound.

### Example 6

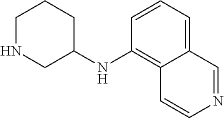 [SMILES: c1cc(NC2CCCNC2)c2ccncc2c1]

### N-(Piperidin-3-yl)isoquinolin-5-amine

Reaction of tert-butyl 3-oxopiperidine-1-carboxylate and isoquinolin-5-amine using the method of Example 3 followed by deprotection using the method of Example 4 afforded the title compound.

### Example 7

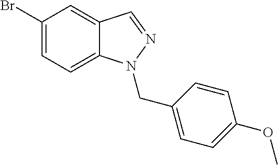 [SMILES: COc1ccc(Cn2ncc3cc(Br)ccc32)cc1]

### 5-Bromo-1-(4-methoxybenzyl)-1H-indazole

To a suspension of 1.1 equivalents of KOtBu in THF was added 1 equivalent 5-bromo-1H-indazole in THF. After 30 min, 4-methoxybenzyl chloride (1.05 equivalents) was added (neat) and the resulting pale yellow solution was stirred 48 h. The reaction was quenched by addition of saturated NH₄Cl solution, and the mixture was extracted with EtOAc. Evaporation of the organic phase followed by column chromatography of the residue on silica gel, eluting with 1/9-EtOAc/heptane, afforded the title compound, which was recrystallized from toluene/heptane (1/5) to afford the title compound as colorless cubes. The N-2 regioisomer was isolated in an equivalent yield.

### Example 8

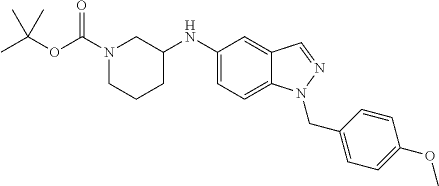 [SMILES: COc1ccc(Cn2ncc3cc(NC4CCCN(C(=O)OC(C)(C)C)C4)ccc32)cc1]

### (S)-tert-Butyl 3-(1-(4-Methoxybenzyl)-1H-indazol-5-ylamino)piperidine-1-carboxylate

To a solution of 5-bromo-1-(4-methoxybenzyl)-1H-indazole (Example 7) in toluene was added, in succession, 1.2 equivalents of (S)-tert-butyl 3-aminopiperidine-1-carboxylate, sodium tert-butoxide (1.8 equivalents), and rac-(±)-BINAP (0.105 equivalents). The flask was evacuated and refilled with nitrogen three times, after which Pd₂dba₃ (1.5 mol %) was added. The flask was again purged with nitrogen three times, and was then heated to 80° C. overnight. The solution was cooled to room temperature and then filtered through a pad of celite, washing with additional toluene. The toluene solution was then loaded directly onto a silica gel column that had been packed with heptane. The column was flushed with 2 column volumes of heptane, and then eluted with 40/60—EtOAc/heptane to afford the title compound.

### Example 9

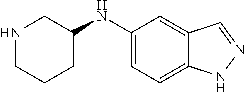

### (S)-N-(Piperidin-3-yl)-1H-indazol-5-amine

A solution of (S)-tert-butyl 3-(1-(4-methoxybenzyl)-1H-indazol-5-ylamino)piperidine-1-carboxylate in excess TFA was stirred at room temperature for 15 min, after which the solvent was evaporated. Chromatography of the residue on silica gel, eluting first with dichloromethane and then with 90:9:1 dichloromethane:MeOH:NH₄OH, afforded the material in which the BOC protecting group had been removed.

The residue thus obtained was then dissolved again in excess TFA, along with 1,3-dimethoxybenzene (2 equivalents) and was heated to reflux overnight. The TFA was removed by evaporation, and the residue was again chromatographed as described above to afford the title compound.

### Example 10

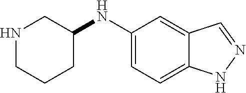

### (R)-N-(Piperidin-3-yl)-1H-indazol-5-amine

Reaction of 5-bromo-1-(4-methoxybenzyl)-1H-indazole and (R)-tert-butyl 3-aminopiperidine-1-carboxylate using the method of Example 8 followed by deprotection using the method of Example 9 afforded the title compound.

### Example 11

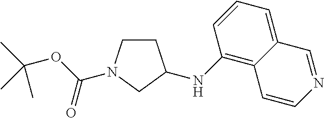 [SMILES: CC(C)(C)OC(=O)N1CCC(Nc2cccc3cnccc23)C1]

### (R)-tert-Butyl 3-(Isoquinolin-5-ylamino)pyrrolidine-1-carboxylate

Into a 50 mL round bottom flask were added equimolar amounts of 5-bromoisoquinoline and (R)-tert-butyl 3-aminopyrrolidine-1-carboxylate, palladium acetate (0.15 equivalents), rac-(±)-(BINAP (0.15 equivalents), and cesium carbonate (1.6 equivalents) in toluene. The vessel was evacuated, refilled with nitrogen and stirred at 80° C. for 12 h. The mixture was diluted with ethyl acetate, washed with water, and the organic phase was dried over MgSO₄, filtered and evaporated to afford the title compound.

### Example 12

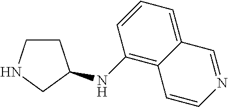 [SMILES: c1cc(N[C@@H]2CCNC2)c2ccncc2c1]

### (R)-N-(Pyrrolidin-3-yl)isoquinolin-5-amine

Deprotection of (R)-tert-butyl 3-(isoquinolin-5-ylamino)pyrrolidine-1-carboxylate following the method of Example 9 afforded the title compound.

### Example 13

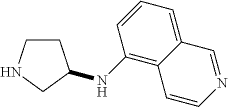

### (S)-N-(Pyrrolidin-3-yl)isoquinolin-5-amine

Reaction of (S)-tert-butyl 3-aminopyrrolidine-1-carboxylate and 5-bromoisoquinoline using the method of Example 11 followed by deprotection using the method of Example 9 afforded the title compound.

Examples 14-46 shows the preparation of pro-drugs Compounds 14-46, respectively.

### Example 14

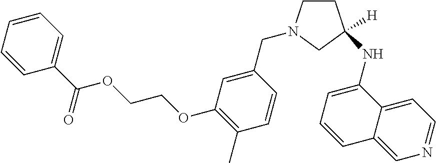 [SMILES: Cc1ccc(CN2CC[C@@H](Nc3cccc4cnccc34)C2)cc1OCCOC(=O)c1ccccc1]

### 2-(5-(((R)-3-isoquinolin-5-ylamino)pyrrolidin-1-yl)methyl)-2-methylphenoxy)ethyl benzoate

A solution of (R)-N-(pyrrolidin-3-yl)isoquinolin-5-amine and an equimolar amount of 2-(5-formyl-2-methylphenoxy)ethyl benzoate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.14 (s, 1H), 8.46 (d, 1H), 8.04 (d, 2H), 7.52-7.59 (m, 2H), 7.38-7.47 (m, 3H), 7.24-7.33 (m, 1H), 7.08 (d, 1H), 6.8-6.88 (m, 2H), 6.69 (d, 1H), 4.6-4.68 (m, 3H), 4.1-4.37 (m, 3H), 3.62 (dd, 2H), 2.8-2.9 (m, 2H), 2.7-2.77 (m, 1 H), 2.36-2.55 (m, 2H), 2.2 (s, 3H), 1.75-1.85 (m, 1H)

### Example 15

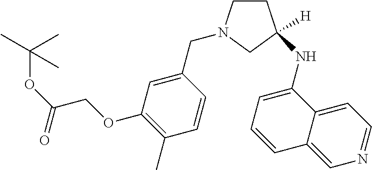 [SMILES: Cc1ccc(CN2CC[C@@H](Nc3cccc4cnccc34)C2)cc1OCC(=O)OC(C)(C)C]

### (R)-tent-butyl 2-(5-((3-isoquinolin-5-ylamino)pyrrolidin-1-yl)methyl)-2-methylphenoxy)acetate

A solution of (R)-N-(pyrrolidin-3-yl)isoquinolin-5-amine and an equimolar amount of tert-butyl 2-(5-formyl-2-methylphenoxy)acetate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.15 (s, 1H), 8.47 (d, 1H), 7.57 (d, 1H), 7.4-7.47 (m, 1H), 7.26-7.34 (m, 1H), 7.1 (d, 1H), 6.82-6.86 (m, 1H), 6.73-6.77 (m, 2H), 4.54-4.62 (m, 3H), 4.1-4.2 (m, 1H), 3.61 (s, 2H), 2.75-2.90 (m, 2H), 2.64-2.72 (m, 1H), 2.35-2.54 (m, 2H), 2.27 (s, 3H), 1.7-1.82 (m, 1H), 1.46 (s, 9H)

### Example 16

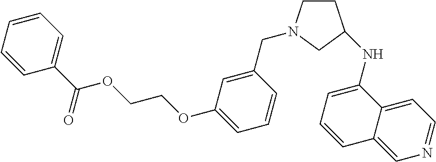 [SMILES: O=C(OCCOc1cccc(CN2CCC(Nc3cccc4cnccc34)C2)c1)c1ccccc1]

### 2-(3-((3-(isoquinolin-5-ylamino)pyrrolidin-1-yl)methyl)phenoxy)ethyl benzoate

A solution of N-(pyrrolidin-3-yl)isoquinolin-5-amine and an equimolar amount of 2-(3-formylphenoxy)ethyl benzoate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound.

### Example 17

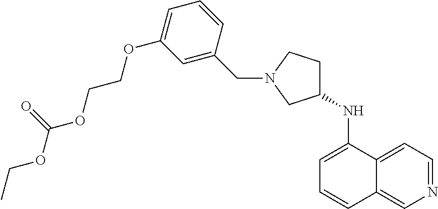

### 2-(3-(((R)-3-(isoquinolin-5-ylamino)pyrrolidin-1-yl)methyl)phenoxy)ethyl ethyl carbonate

A solution of (R)-N-(pyrrolidin-3-yl)isoquinolin-5-amine and an equimolar amount of ethyl 2-(3-formylphenoxy)ethyl carbonate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CD₃OD) δ 9.1 (s, 1H), 8.37 (d, 1H), 8.04 (d, 1H), 7.35-7.56 (m, 3H), 7.02-7.12 (m, 3H), 6.84 (d, 1H), 4.41-4.5 (m, 5H), 4.13-4.21 (m, 4H), 3.56-3.8 (m, 2H), 3.4-3.53 (m, 2H), 2.6-2.72 (m, 1H), 2.21-2.34 (m, 1H), 1.26 (t, 3H)

### Example 18

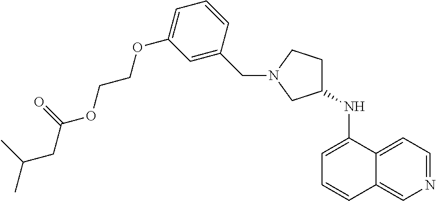

### 2-(3-((((R)-3-(isoquinolin-5-ylamino)pyrrolidin-1-yl)methyl)phenoxy)ethyl 3-methylbutanoate)

A solution of (R)-N-(pyrrolidin-3-yl)isoquinolin-5-amine and an equimolar amount of ethyl 2-(3-formylphenoxy)ethyl 3-methylbutanoate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CD₃OD) δ 9.33 (s, 1H), 8.38-8.45 (m, 2H), 7.58-7.71 (m, 2H), 7.36-7.42 (m, 1H), 7.02-7.2 (m, 4H), 4.39-4.6 (m, 5H), 4.16-4.23 (m, 2H), 3.4-3.9 (4H), 3.54-3.76 (m, 1H), 2.24-2.38 (m, 1H), 2.21 (d, 2H), 1.96-2.1 (m, 1H), 0.93 (d, 6H)

### Example 19

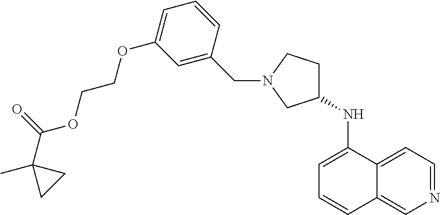

### 2-(3-(((R)-3-(isoquinolin-5-ylamino)pyrrolidin-1-yl)methyl)phenoxy)ethyl 1-methylcyclopropanecarboxylate

A solution of (R)-N-(pyrrolidin-3-yl)isoquinolin-5-amine and an equimolar amount of 2-(3-formylphenoxy)ethyl-1-methylcyclopropane carboxylate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CD₃OD) δ 9.12 (s, 1H), 8.39 (d, 1H), 8.02 (d, 1H), 7.35-7.56 (m, 3H), 7.01-7.13 (m, 3H), 6.82-6.86 (m, 1H), 4.33-4.52 (m, 5H), 4.12-4.2 (m, 2H), 3.38-3.8 (m, 4H), 2.58-2.73 (m, 1H), 2.22-2.34 (m, 1H), 1.24 (s, 3H), 1.13-1.18 (m, 2H), 0.65-0.72 (m, 2H)

### Example 20

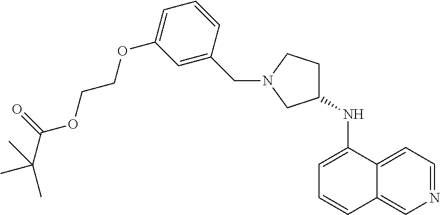

### 2-(3-(((R)-3-(isoquinolin-5-ylamino)pyrrolidin-1-yl)methyl)phenoxy)ethyl pivalate

A solution of (R)-N-(pyrrolidin-3-yl)isoquinolin-5-amine and an equimolar amount of 2-(3-formylphenoxy)ethyl pivalate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CD₃OD) δ 9.27 (s, 1H), 8.42 (d, 1H), 8.29 (d, 1H), 7.55-7.65 (m, 2H), 7.36-7.42 (m, 1H), 6.95-7.18 (m, 4H), 4.35-4.58 (m, 5H), 4.15-4.23 (m, 2H), 3.42-3.9 (m, 4H), 2.55-2.78 (m, 1H), 2.23-2.36 (m, 1H), 1.17 (s, 9H)

### Example 21

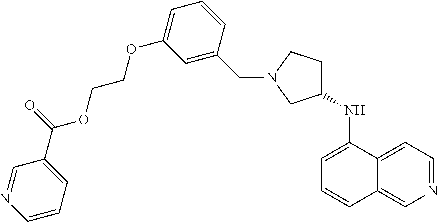

### 2-(3-(((R)-3-(isoquinolin-5-ylamino)pyrrolidin-1-yl)methyl)phenoxy)ethyl nicotinate

A solution of (R)-N-(pyrrolidin-3-yl)isoquinolin-5-amine and an equimolar amount of 2-(3-formylphenoxy)ethyl nicotinate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.2 (s, 1H), 9.06 (s, 1H), 8.73(d, 1H), 8.35-8.42 (m, 2H), 8.13 (d, 1H), 7.48-7.6 (m, 3H), 7.35-7.43 (m, 1H), 7.08-7.16 (m, 3H), 6.9 (d, 1H), 4.66-4.73 (m, 2H), 4.3-4.55 (m, 5H), 3.4-3.8 (m, 4H), 2.55-2.8 (m, 1H), 2.25-2.36 (m, 1H)

### Example 22

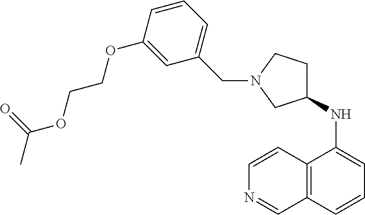 [SMILES: CC(=O)OCCOc1cccc(CN2CC[C@@H](Nc3cccc4cnccc34)C2)c1]

### 2-(3-(((R)-3-(isoquinolin-5-ylamino)pyrrolidin-1-yl)methyl)phenoxy)ethyl benzoate

A solution of (R)-N-(pyrrolidin-3-yl)isoquinolin-5-amine and an equimolar amount of 2-(3-formylphenoxy)ethyl benzoate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound.

### Example 23

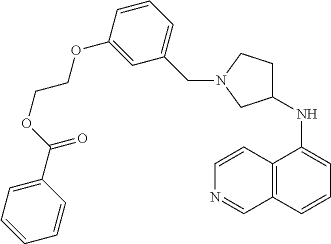 [SMILES: O=C(OCCOc1cccc(CN2CCC(Nc3cccc4cnccc34)C2)c1)c1ccccc1]

### 2-(3-((3-(isoquinolin-5-ylamino)pyrrolidin-1-yl)methyl)phenoxy)ethyl benzoate

A solution of N-(pyrrolidin-3-yl)isoquinolin-5-amine and an equimolar amount of 2-(3-formylphenoxy)ethyl benzoate in DMSO was treated with a twofold excess of sodium triacetoxyborohydride for 18 hours. The reaction was monitored by HPLC for complete conversion of the starting materials to the product and when complete, was quenched with acetonitrile. Evaporation afforded a residue which was chromatographed on C18 silica gel to yield the title compound. ¹H NMR (CDCl₃) δ 9.14 (s, 1H), 8.45 (d, 1H), 8.04-8.07 (m, 2H), 7.64 (d, 1H), 7.5-7.6 (m, 1H), 7.4-7.45 (m, 3H), 7.2-7.35 (m, 3H), 6.93-7.0 (m, 2H), 6.87 (dd, 1H), 6.68 (d, 1H), 4.6-4.7 (m, 2H), 4.25-4.35 (m, 2H), 3.71 (s, 2H), 2.8-3.05 (m, 3H)), 2.38-2.65 (m, 3H), 1.8-1.93 (m, 1H)

### Example 24

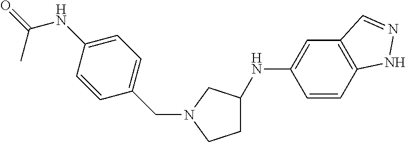 [SMILES: CC(=O)Nc1ccc(CN2CCC(Nc3ccc4[nH]ncc4c3)C2)cc1]

### N-(4-((3-(1H-indazol-5-ylamino)pyrrolidin-1-yl)methyl)phenyl)acetamide

A solution of 2,2-dimethyl-1-(5-(pyrrolidin-3-ylamino)-1H-indazol-1-yl)propan-1-one and an equimolar amount of N-(4-formylphenyl)acetamide in DCE was treated with equimolar amounts of glacial acetic acid and sodium triacetoxyborohydride. The reaction was monitored by HPLC for complete conversion of the starting materials to the product, and when complete, was quenched with equal volumes of aqueous sodium bicarbonate and acetonitrile. The organic layer was separated and washed with dilute aqueous HCl, NaHCO₃, and brine, and dried over MgSO₄. Evaporation afforded a residue which was chromatographed on C18 silica gel to yield the a solid which was dissolved in MeOH and treated with 3 equivalents of sodium methoxide until the starting material was consumed as monitored by HPLC. The mixture was diluted with ethyl acetate and washed with water. The organic phase was separated, dried over MgSO₄, filtered and evaporated to dryness to afford the title compound. ¹H NMR (CDCl₃) δ 7.90 (s, 1H), 7.43 (d, 2H), 7.23-7.35 (m, 5H), 6.78 (d, 2H), 4.02 (br s, 1H), 3.60 (dd, 2H), 2.70-2.85 (m, 2H), 2.58-2.63 (m, 1H), 2.25-2.5 (m, 2H), 2.16 (s, 3H), 1.65-1.75 (m, 2H)

### Example 25

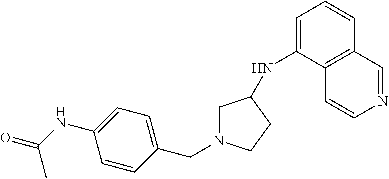 [SMILES: CC(=O)Nc1ccc(CN2CCC(Nc3cccc4cnccc34)C2)cc1]

### N-(4-((3-(isoquinolin-5-ylamino)pyrrolidin-1-yl)methyl)phenyl)acetamide

A solution of N-(pyrrolidin-3-yl)isoquinolin-5-amine and an equimolar amount of 4-acetamidobenzaldehyde in THF was treated with equimolar amounts of glacial acetic acid and sodium triacetoxyborohydride. The reaction was monitored by HPLC for complete conversion of the starting materials to the product, and when complete, was quenched with equal volumes of aqueous sodium bicarbonate and acetonitrile. The organic layer was separated and washed with dilute aqueous HCl, aHCO₃, and brine, and dried over MgSO₄. Evaporation afforded a residue which was chromatographed on C18 silica gel to yield the title compound. ¹H NMR (CDCl₃) δ 9.15 (s, 1H), 8.45 (d, 1H), 7.20-7.65 (m, 8H), 7.65 (d, 1H), 4.63 (br d, 1H), 4.05-4.2 (m, 1H), 3.62 (s, 2H), 2.65-2.9 (m, 3H), 2.35-2.55 (m, 2H), 2.16 (s, 3H), 1.7-1.9 (m, 1H)

### Example 26

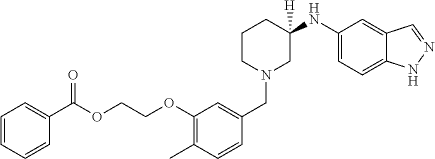 [SMILES: Cc1ccc(CN2CCC[C@@H](Nc3ccc4[nH]ncc4c3)C2)cc1OCCOC(=O)c1ccccc1]

### 2-(5-(((R)-3-(1H-indazol-5-ylamino)piperidin-1-yl)methyl)-2-methylphenoxy)ethyl benzoate

A solution of (R)-N-(piperidin-3-yl)-1H-indazol-5-amine dihydrochloride and an equimolar amount of 2-(5-formyl-2-methylphenoxy)ethyl benzoate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.80 (s, 1H), 8.06 (d, 2H), 7.85 (s, 1H), 7.51-7.60 (m, 1H), 7.38-7.45 (m, 2H), 7.23-7.28 (m, 2H), 7.04-7.08 (m, 1H), 6.77-6.88 (m, 4H), 4.68-4.74 (m, 2H), 4.25-4.35 (m, 2H), 3.98 (br s, 1H), 3.50-3.62 (m, 1H), 2.70-2.77 (m, 1H), 2.30-2.48 (m, 3H), 2.20 (s, 3H), 1.50-1.80 (m, 5H)

### Example 27

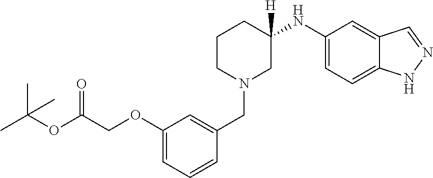 [SMILES: CC(C)(C)OC(=O)COc1cccc(CN2CCC[C@H](Nc3ccc4[nH]ncc4c3)C2)c1]

### tert-Butyl 2-(3-(((S)-3-(1H-indazol-5-ylamino)piperidin-1-yl)methyl)phenoxy) acetate

A solution of (S)-N-(piperidin-3-yl)-1H-indazol-5-amine dihydrochloride and a 1.5 molar excess of tert-butyl 2-(3-formylphenoxy)acetate in THF containing 3 equivalents of glacial acetic acid was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 7.87 (s, 1H), 7.19-7.32 (m, 3H), 6.92-6.97 (m, 2H), 6.75-6.84 (m, 3H), 4.52 (s, 2H), 3.5-3.65 (m, 3H), 2.7-2.83 (m, 1H), 2.26-2.48 (m, 3H), 1.48-1.84 (m, 14H)

### Example 28

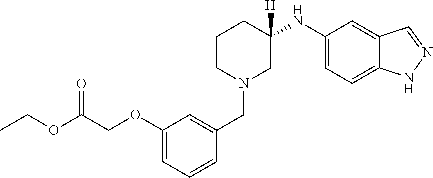 [SMILES: CCOC(=O)COc1cccc(CN2CCC[C@H](Nc3ccc4[nH]ncc4c3)C2)c1]

### Ethyl 2-(3-(S)-3-(1H-indazol-5-ylamino)piperidin-1-yl)methyl)phenoxy)acetate

A solution of (S)-N-(piperidin-3-yl)-1H-indazol-5-amine dihydrochloride and a 1.5 molar excess of ethyl 2-(3-formylphenoxy)acetate in THF containing 3 equivalents of glacial acetic acid was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.8 (br s, 1H), 7.86 (s, 1H), 7.2-7.26 (m, 2H), 6.77-7.0 (m, 5H), 4.63 (s, 2H), 4.29 (q, 2H), 3.44-3.64 (m, 3H), 2.72-2.80 (m, 1H), 2.3-2.45 (m, 3H), 1.5-1.8 (m, 5H), 1.29 (t, 3H)

### Example 29

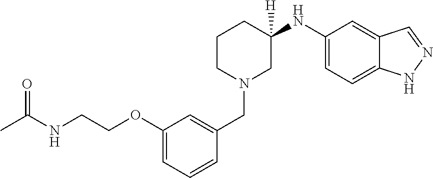 [SMILES: CC(=O)NCCOc1cccc(CN2CCC[C@@H](Nc3ccc4[nH]ncc4c3)C2)c1]

### N-(2-(3-(((R)-3-(1H-indazol-5-ylamino)piperidin-1-yl)methyl)phenoxy)ethyl) acetamide

An equimolar solution of (R)-N-(piperidin-3-yl)-1H-indazol-5-amine dihydrochloride and N-(2-(3-formylphenoxy)ethyl)acetamide in MeOH containing a twofold molar excess of sodium acetate was treated with a 1.5 molar excess of sodium cyanoborohydride for 18 hours. The reaction was monitored by HPLC for complete conversion of the starting materials to the product and when complete, was quenched with aqueous sodium bicarbonate. The solution was extracted with ethyl acetate, washed with dilute HCl and brine then dried over MgSO₄. Evaporation afforded a residue which was chromatographed on silica gel to yield the title compound. ¹H NMR (CDCl₃) δ 7.85 (s, 1H), 7.2-7.33 (m, 2H), 6.75-6.94 (m, 5H), 5.95 (br s, 1H), 3.97-4.04 (m, 2H), 3.4-3.66 (m, 6H), 2.75 (br d, 1H), 2.28-2.5 (m, 3H), 2.0 (s, 3H), 1.5-1.8 (m, 4H)

### Example 30

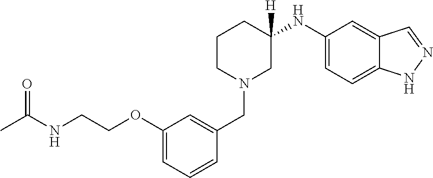 [SMILES: CC(=O)NCCOc1cccc(CN2CCC[C@H](Nc3ccc4[nH]ncc4c3)C2)c1]

### N-(2-(3-(((S)-3-(1H-indazol-5-ylamino)piperidin-1-yl)methyl)phenoxy)ethyl) acetamide

A solution of (S)-N-(piperidin-3-yl)-1H-indazol-5-amine dihydrochloride and an equimolar amount of N-(2-(3-formylphenoxy)ethyl)acetamide in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.85 (br s, 1H), 7.86 (s, 1H), 7.2-7.32 (m, 2H), 6.75-6.95(5H), 5.9 (bra s, 1H), 3.98-4.06 (m, 2H), 3.42-3.7 (m, 6H), 2.68-2.75 (m, 1H), 2.25-2.48 (m, 3H), 2.0 (s, 3H), 1.65-1.8 (m, 2H), 1.6 (m, 2H, hidden under water peak)

### Example 31

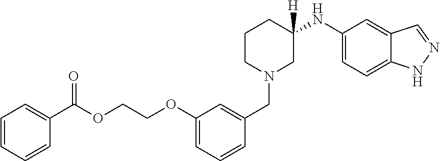 [SMILES: O=C(OCCOc1cccc(CN2CCC[C@H](Nc3ccc4[nH]ncc4c3)C2)c1)c1ccccc1]

### 2-(3-(((S)-3-(1H-indazol-5-ylamino)piperidin-1-yl)methyl)phenoxy)ethyl benzoate

A solution of (S)-N-(piperidin-3-yl)-1H-indazol-5-amine dihydrochloride and an equimolar amount of 2-(3-formylphenoxy)ethyl benzoate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.85 (br s, 1H), 8.04-8.08 (m, 2H), 7.86 (d, 1H), 7.52-7.59 (m, 1H), 7.38-7.46 (m, 2H), 7.2-7.3 (m, 2H), 6.91-6.97 (m, 2H), 6.79-6.84 (m, 3H), 4.65-4.70 (m, 2H), 4.28-4.34 (m, 2H), 3.45-3.65 (m, 3H), 2.67-2.78 (m, 1H), 2.27-2.45 (m, 3H), 1.50-1.78 (m, 5H)

### Example 32

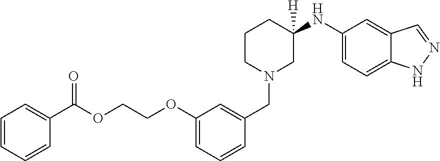 [SMILES: O=C(OCCOc1cccc(CN2CCC[C@@H](Nc3ccc4[nH]ncc4c3)C2)c1)c1ccccc1]

### 2-(3-((R)-3-(1H-indazol-5-ylamino)piperidin-1-yl)methyl)phenoxy)ethyl benzoate

A solution of (R)-N-(piperidin-3-yl)-1H-indazol-5-amine dihydrochloride and an equimolar amount of 2-(3-formylphenoxy)ethyl benzoate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.85 (br s, 1H), 8.04-8.08 (m, 2H), 7.86 (d, 1H), 7.52-7.59 (m, 1H), 7.38-7.46 (m, 2H), 7.2-7.3 (m, 2H), 6.91-6.97 (m, 2H), 6.79-6.84 (m, 3H), 4.65-4.70 (m, 2H), 4.28-4.34 (m, 2H), 3.45-3.65 (m, 3H), 2.67-2.78 (m, 1H), 2.27-2.45 (m, 3H), 1.50-1.78 (m, 5H)

### Example 33

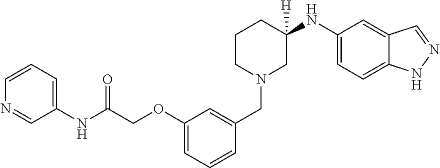 [SMILES: O=C(COc1cccc(CN2CCC[C@@H](Nc3ccc4[nH]ncc4c3)C2)c1)Nc1cccnc1]

### 2-(3-(((R)-3-(1H-indazol-5-ylamino)piperidin-1-yl)methyl)phenoxy)-N-(pyridin-3-yl)acetamide

An equimolar solution of (R)-N-(piperidin-3-yl)-1H-indazol-5-amine dihydrochloride and 2-(3-formylphenoxy)-N-(pyridin-3-yl)acetamide in MeOH containing a twofold molar excess of sodium acetate was treated with a 1.5 molar excess of sodium cyanoborohydride for 18 hours. The reaction was monitored by HPLC for complete conversion of the starting materials to the product and when complete, was quenched with aqueous sodium bicarbonate. The solution was extracted with ethyl acetate, washed with dilute HCl and brine then dried over MgSO₄.

Evaporation afforded a residue which was chromatographed on silica gel to yield the title compound. ¹H NMR (CDCl₃) δ 8.64 (d, 1H), 8.30-8.41 (m, 2H), 8.2-8.24 (m, 1H), 7.85 (s, 1H), 7.25-7.3 (m, 3H), 7-7.05(2H), 6.8-6.9 (m, 3H), 4.64 (s, 2H), 3.45-3.62 (m, 3H), 2.75 (br d, 1H), 2.2-2.5 (m, 4H), 1.45-1.8 (m, 6H)

Example 34

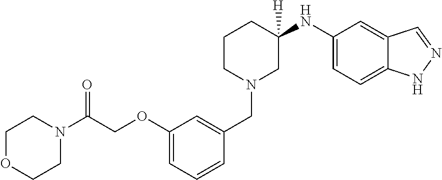 [SMILES: O=C(COc1cccc(CN2CCC[C@@H](Nc3ccc4[nH]ncc4c3)C2)c1)N1CCOCC1]

### 2-(3-(((R)-3-(1H-indazol-5-ylamino)piperidin-1-yl)methyl)phenoxy)-1-morpholinoethanone

An equimolar solution of (R)-N-(piperidin-3-yl)-1H-indazol-5-amine dihydrochloride and 3-(2-(morpholin-1-yl)-2-oxoethoxy)benzaldehyde in MeOH containing a twofold molar excess of sodium acetate was treated with a 1.5 molar excess of sodium cyanoborohydride for 18 hours. The reaction was monitored by HPLC for complete conversion of the starting materials to the product and when complete, was quenched with aqueous sodium bicarbonate. The solution was extracted with ethyl acetate, washed with dilute HCl and brine then dried over MgSO₄.

Evaporation afforded a residue which was chromatographed on silica gel to yield the title compound. ¹H NMR (CDCl₃) δ 7.86 (s, 1H), 7.2-7.32 (m, 2H), 6.78-7.0 (m, 5H), 4.69 (s, 2H), 3.4-3.68 (m, 11H), 2.72 (br d, 1H), 2.3-2.5 (m, 3H), 2.4-2.8 (m, 5H)

### Example 35

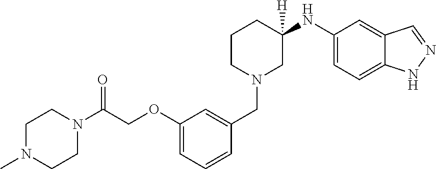 [SMILES: CN1CCN(C(=O)COc2cccc(CN3CCC[C@@H](Nc4ccc5[nH]ncc5c4)C3)c2)CC1]

### 2-(3-(((R)-3-(1H-indazol-5-ylamino)piperidin-1-yl)methyl)phenoxy)-1-(4-methylpiperazin-1-yl)pethanone

An equimolar solution of (R)-N-(piperidin-3-yl)-1H-indazol-5-amine dihydrochloride and 3-(2-(4-methylpiperazin-1-yl)-2-oxoethoxy)benzaldehyde in MeOH containing a twofold molar excess of sodium acetate was treated with a 1.5 molar excess of sodium cyanoborohydride for 18 hours. The reaction was monitored by HPLC for complete conversion of the starting materials to the product and when complete, was quenched with aqueous sodium bicarbonate. The solution was extracted with ethyl acetate, washed with dilute HCl and brine then dried over MgSO₄. Evaporation afforded a residue which was chromatographed on silica gel to yield the title compound.

### Example 36

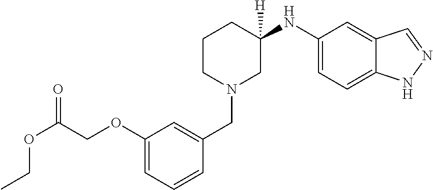 [SMILES: CCOC(=O)COc1cccc(CN2CCC[C@@H](Nc3ccc4[nH]ncc4c3)C2)c1]

### Ethyl 2-(3-(((R)-3-(1H-indazol-4-ylamino)piperidin-1-yl)methyl)phenoxy)acetate

A solution of (R)-N-(piperidin-3-yl)-1H-indazol-5-amine dihydrochloride and an equimolar amount of ethyl 2-(3-formylphenoxy)acetate in THF was treated with a twofold molar excess of sodium acetate and sodium triacetoxyborohydride. The reaction was monitored by HPLC for complete conversion of the starting materials to the product, and when complete, was quenched with equal volumes of aqueous sodium bicarbonate and acetonitrile. The organic layer was separated and washed with dilute aqueous HCl, NaHCO₃, and brine, and dried over MgSO₄. Evaporation afforded a residue which was chromatographed on silica gel to yield the title compound. ¹H NMR (CDCl₃) δ 7.87 (s, 1H), 7.2-7.33 (m, 3H), 6.92-6.98 (m, 2H), 6.73-6.85 (m, 3H), 4.62 (s, 2H), 4.27 (q, 2H), 3.42-3.64 (m, 3H), 2.7-2.8 (m, 1H), 2.28-2.43 (m, 3H), 1.52-1.78 (m, 4H), 1.29 (t, 3H)

### Example 37

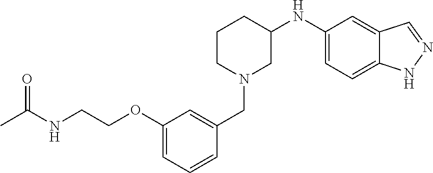 [SMILES: CC(=O)NCCOc1cccc(CN2CCCC(Nc3ccc4[nH]ncc4c3)C2)c1]

### N-(2-(3-((3-(1H-indazol-5-ylamino)piperidin-1-yl)methyl)phenoxy)ethyl)acetamide

A solution of 2,2-dimethyl-1-(5-(piperidin-3-ylamino)-1H-indazol-1-yl)propan-1-one and an equimolar amount of N-(2-(3-formylphenoxy)ethyl)acetamide in THF was treated with a twofold excess of sodium triacetoxyborohydride for 18 hours. The reaction was monitored by HPLC for complete conversion of the starting materials to the product and when complete, was quenched with aqueous sodium bicarbonate. The solution was extracted with ethyl acetate, washed with dilute HCl and brine then dried over MgSO₄. Evaporation afforded a residue which was dissolved in MeOH and treated with an excess of K₂CO₃ for 18 hours. The MeOH was decanted and evaporated to a residue which was chromatographed on C18 silica gel to yield the title compound.

### Example 38

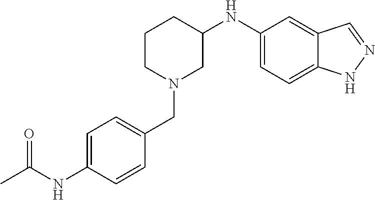 [SMILES: CC(=O)Nc1ccc(CN2CCCC(Nc3ccc4[nH]ncc4c3)C2)cc1]

### N-(4-((3-(1H-indazol-5-ylamino)piperidin-1-yl)methyl)phenyl)acetamide

A solution of 2,2-dimethyl-1-(5-(piperidin-3-ylamino)-1H-indazol-1-yl)propan-1-one and an equimolar amount of N-(4-formylphenyl)acetamide in DCE was treated with equimolar amounts of glacial acetic acid and sodium triacetoxyborohydride. The reaction was monitored by HPLC for complete conversion of the starting materials to the product, and when complete, was quenched with equal volumes of aqueous sodium bicarbonate and acetonitrile. The organic layer was separated and washed with dilute aqueous HCl, NaHCO₃, and brine, and dried over MgSO₄. Evaporation afforded a residue which was chromatographed on C18 silica gel to yield a solid which was dissolved in MeOH and treated with 3 equivalents of sodium methoxide until the starting material was consumed as monitored by HPLC. The mixture was diluted with ethyl acetate and washed with water. The organic phase was separated, dried over MgSO₄, filtered and evaporated to dryness to afford the title compound. ¹H NMR (CDCl₃) δ 7.85 (s, 1H), 7.45 (d, 2H), 7.22-7.32 (m, 5H), 6.80 (d, 2H), 3.58 (br s, 1H), 3.48(dd, 2H), 2.68-2.75 (m, 1H), 2.25-2.42 (m, 3H), 2.17 (s, 3H), 1.5-1.8 (m, 5H)

### Example 39

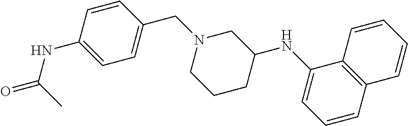 [SMILES: CC(=O)Nc1ccc(CN2CCCC(Nc3cccc4ccccc34)C2)cc1]

### N-(4-((3-(isoquinolin-5-ylamino)piperidin-1-yl)methyl)phenyl)acetamide

A solution of N-(piperidin-3-yl)isoquinolin-5-amine and an equimolar amount of N-(3-formylphenyl)acetamide in THF was treated with equimolar amounts of glacial acetic acid and sodium triacetoxyborohydride. The reaction was monitored by HPLC for complete conversion of the starting materials to the product, and when complete, was quenched with equal volumes of aqueous sodium bicarbonate and acetonitrile. The organic layer was separated and washed with dilute aqueous HCl, NaHCO₃, and brine, and dried over MgSO₄. Evaporation afforded a residue which was chromatographed on C18 silica gel to yield the title compound. ¹H NMR (CDCl₃) δ 9.15 (s, 1H), 8.45 (d, 2H), 7.2-7.6 (m, 7H), 6.7 (d, 2H), 5.05 (br s, 1H), 3.8 (br s, 1H), 3.5 (dd, 2H), 2.45-2.63 (m, 3H), 2.28-2.42 (m, 1H), 2.15 (s, 3H), 1.50-1.85 (m, 5H)

Example 40

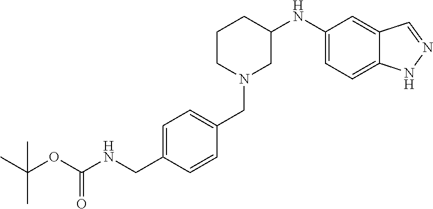 [SMILES: CC(C)(C)OC(=O)NCc1ccc(CN2CCCC(Nc3ccc4[nH]ncc4c3)C2)cc1]

### tent-Butyl (3-((3-(1H-indazol-5-ylamino)piperidin-1-yl)methyl)phenyl)methyl carbamate

An equimolar solution of (R)-N-(piperidin-3-yl)-1H-indazol-5-amine dihydrochloride and tert-butyl 3-formylbenzylcarbamate in MeOH containing a twofold molar excess of sodium acetate was treated with a 1.5 molar excess of sodium cyanoborohydride for 18 hours. The reaction was monitored by HPLC for complete conversion of the starting materials to the product and when complete, was quenched with aqueous sodium bicarbonate. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.84 (br s, 1H), 7.86 (s, 1H), 7.15-7.31 (m, 5H), 6.80-6.85 (m, 2H), 4.8 (s, 1H), 4.28-4.32 (d, 2H), 3.95-4.05 (s, 1H), 3.40-3.62 (m, 2H), 2.60-2.74 (s, 1H), 2.14-2.45 (m, 2H), 1.50-1.80 (m, 6H), 1.47 (s, 9H)

### Example 41

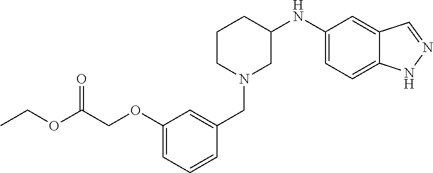 [SMILES: CCOC(=O)COc1cccc(CN2CCCC(Nc3ccc4[nH]ncc4c3)C2)c1]

### Ethyl 2-(3-((3-(1H-indazol-5-ylamino)piperidin-1-yl)methyl)phenoxy)acetate

An equimolar solution of N-(piperidin-3-yl)-1H-indazol-5-amine dihydrochloride and ethyl 2-(3-formylphenoxy)acetate) in 1:1 MeOH/dichloroethane containing an equimolar amount of glacial acetic acid was treated with a 1.3 molar excess of sodium cyanoborohydride for 18 hours. The reaction was monitored by HPLC for complete conversion of the starting materials to the product and when complete, was quenched with aqueous sodium bicarbonate. The solution was extracted with ethyl acetate, washed with dilute HCl and brine then dried over MgSO₄. Evaporation afforded a residue which was chromatographed on C18 silica gel to yield the title compound.

### Example 42

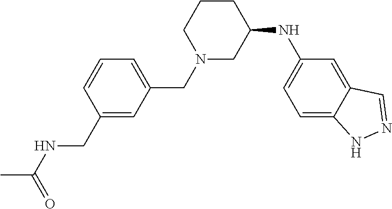 [SMILES: CC(=O)NCc1cccc(CN2CCC[C@@H](Nc3ccc4[nH]ncc4c3)C2)c1]

### N-((3-(((R)-3-(1H-indazol-5-ylamino)piperidin-1-yl)methyl)phenyl)methyl) acetamide

An equimolar solution of (R)-N-(piperidin-3-yl)-1H-indazol-5-amine dihydrochloride and N-(3-formylbenzyl)acetamide in MeOH containing a twofold molar excess of sodium acetate was treated with a 1.5 molar excess of sodium cyanoborohydride for 18 hours. The reaction was monitored by HPLC for complete conversion of the starting materials to the product and when complete, was quenched with aqueous sodium bicarbonate. The solution was extracted with ethyl acetate, washed with dilute HCl and brine then dried over MgSO₄. Evaporation afforded a residue which was chromatographed on silica gel to yield the title compound. ¹H NMR (CDCl₃) δ 7.86 (s, 1H), 7.14-7.31 (m, 6H), 6.78-6.85 (m, 2H), 5.65 (br s, 1H), 4.05 (d, 2H), 3.4-3.65 (m, 3H), 2.66-2.74 (m, 1H), 2.16-2.26 (m, 3H), 2.0 (s, 3H), 1.5-1.8 (m, 4H)

### Example 43

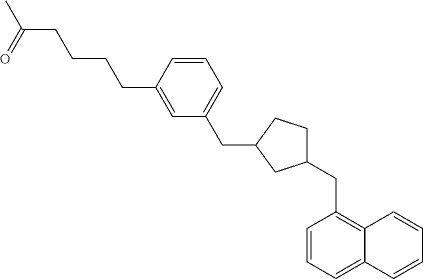 [SMILES: CC(=O)CCCCc1cccc(CC2CCC(Cc3cccc4ccccc34)C2)c1]

### tert-Butyl (4-(((S)-3-(1H-indazol-5-ylamino)piperidin-1-yl)methyl)phenyl) methylcarbamate

A solution of (S)-N-(piperidin-3-yl)-1H-indazol-5-amine dihydrochloride and an equimolar amount of (4-formylbenzyl)carbamic acid tert-butyl ester in THF was treated with equimolar amounts of glacial acetic acid and sodium triacetoxyborohydride. The reaction was monitored by HPLC for complete conversion of the starting materials to the product, and when complete, was quenched with equal volumes of aqueous sodium bicarbonate and acetonitrile. The organic layer was separated and washed with dilute aqueous HCl, NaHCO₃, and brine, and dried over MgSO₄. Evaporation afforded a residue which was chromatographed on silica gel to yield the title compound. ¹H NMR (CDCl₃) δ 9.85 (br s, 1H), 7.86 (s, 1H), 7.26-7.32 (m, 3H), 7.17-7.24(m, 2H), 6.79-6.84 (m, 2H), 4.8 (br s, 1H), 4.29 (br d, 2H), 3.4-3.63 (m, 3H), 2.63-2.77 (m, 1H), 2.28-2.34 (m, 3H), 1.55-1.8 (m, 4H), 1.47 (s, 9H)

### Example 44

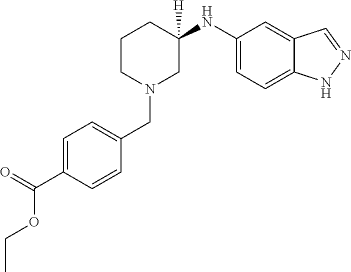 [SMILES: CCOC(=O)c1ccc(CN2CCC[C@@H](Nc3ccc4[nH]ncc4c3)C2)cc1]

### Ethyl 4-(((R)-3-(1H-indazol-5-ylamino)piperidin-1-yl)methyl)benzoate

An equimolar solution of (R)-N-(piperidin-3-yl)-1H-indazol-5-amine dihydrochloride and ethyl 4-formylbenzoate in MeOH containing a twofold molar excess of sodium acetate was treated with a 1.5 molar excess of sodium cyanoborohydride for 18 hours. The reaction was monitored by HPLC for complete conversion of the starting materials to the product and when complete, was quenched with aqueous sodium bicarbonate. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.87 (br s, 1H), 7.99 (d, 2H), 7.86 (s, 1H), 7.41 (d, 2H), 7.26-7.33 (m, 1H), 6.76-6.84 (m, 2H), 4.37 (q, 2H), 3.46-3.62 (m, 4H), 2.75 (br d, 1H), 2.26-2.43 (m, 3H), 1.5-1.8 (m, 4H), 1.42 (t, 3H)

### Example 45

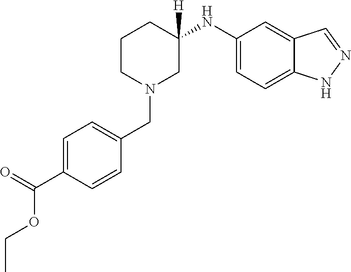 [SMILES: CCOC(=O)c1ccc(CN2CCC[C@H](Nc3ccc4[nH]ncc4c3)C2)cc1]

### Ethyl 4-(((S)-3-(1H-indazol-5-ylamino)piperidin-1-yl)methyl)benzoate

A solution of (S)-N-(piperidin-3-yl)-1H-indazol-5-amine dihydrochloride and an equimolar amount of ethyl 4-formylbenzoate in THF was treated with a twofold excess of sodium triacetoxyborohydride for 18 hours. The reaction was monitored by HPLC for complete conversion of the starting materials to the product and when complete, was quenched with aqueous NaOH. The solution was extracted with ethyl acetate, washed with dilute HCl and brine then dried over MgSO₄. Evaporation afforded a residue which was chromatographed on silica gel to yield the title compound. ¹H NMR (CDCl₃) δ 9.82 (br s, 1H), 7.99 (d, 2H), 7.86 (s, 1H), 7.40 (d, 2H), 7.22-7.32 (m, 1H), 6.8-6.85 (m, 2H), 4.37 (q, 2H), 3.52-3.629m, 3H), 2.7-2.8 (m, 1H), 2.26-2.42 (m, 3H), 1.45-1.8 (m, 5H), 1.39 (t, 3H)

### Example 46

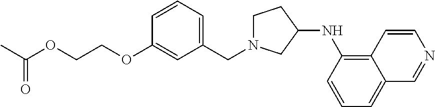 [SMILES: CC(=O)OCCOc1cccc(CN2CCC(Nc3cccc4cnccc34)C2)c1]

### 2-(3-((3-(isoquinolin-5-ylamino)pyrrolidin-1-yl)methyl)phenoxy)ethyl acetate

A solution of N-(pyrrolidin-3-yl)isoquinolin-5-amine and an equimolar amount of 2-(3-formylphenoxy)ethyl acetate in DMSO was treated with a twofold excess of sodium triacetoxyborohydride for 18 hours. The reaction was monitored by HPLC for complete conversion of the starting materials to the product and when complete, was quenched with acetonitrile. Evaporation afforded a residue which was chromatographed on C18 silica gel to yield the title compound. ¹H NMR (CDCl₃) δ 9.15 (s, 1H), 8.46 (d, 1H), 7.71 (d, 1H), 7.2-7.5 (m, 3H), 7.3-7.5 (m, 3H), 6.66 (d, 1H), 4.38-4.46 (m, 2H), 4.14-4.3 (m, 3H), 3.77(dd, 2H), 2.86-3.18 (m, 3H), 2.4-2.66 (m, 2H), 2.09 (s, 3H), 1.84-2.02 (m, 1H)

### Example 47

### Rho Kinase Inhibition Assay

Inhibition of ROCK2 and ROCK1 activity was determined using the IMAP™ Screening Express Kit (Molecular Devices product number #8073). ROCK2 enzyme (Upstate/Chemicon #14-451), ROCK1 (Upstate/Chemicon #14-601) and Fluorescein tagged substrate peptide Fl-AKRRRLSSLRA (Molecular Devices product number R7184) was pre-incubated with a test compound for 5 minutes in buffer containing 10 mM Tris-HCl pH 7.2, 10 mM MgCl₂, and 0.1% BSA. Following the pre-incubation, 10 μM ATP was added to initiate the reaction. After 60 minutes at room temperature, Molecular Devices IMAP™ binding solution was added to bind phosphorylated substrate. After 30 minutes of incubation in the presence of the IMAP™ beads, the fluorescence polarization was read and the ratio was reported as mP. IC₅₀ values for compounds and EC₅₀ values for ATP were calculated using the Prism software from Graphpad, and the results are summarized in Table 1.

This assay demonstrates a compound's ability to inhibit ROCK2 in an in vitro setting using the isolated enzyme. Most of the compounds studied inhibited ROCK2 with an IC₅₀ below many of these inhibiting below 1 The most potent compounds in this assay showed IC₅₀ values below 250 nM. Compounds having ROCK2 IC₅₀ values on the order of 2 μM or below have been shown to possess efficacy in numerous studies using in vivo models of the disease processes described in this application, specifically in models of elevated TOP and glaucoma. See Tian et al., Arch. Ophthalmol. 116: 633-643, 1998; Tian et al., Invest. Ophthalmol. Vis. Sci. 40: 239-242, 1999; Tian, et al., Exp. Eye Res. 68: 649-655; 1999; Sabanay, et al., Arch. Ophthalmol. 118: 955-962, 2000; Volberg, et al., Cell Motil. Cytoskel. 29: 321-338, 1994; Tian, et al., Exp. Eye Res. 71: 551-566, 2000; Tokushige, et al., Invest. Ophthalmol. Vis. Sci. 48: 3216-3222, 2007; Honjo, et al., Invest. Ophthalmol. Vis. Sci. 42: 137-144, 2001.

Compounds 14-46 were prepared according to Examples 14-46. The structures of parent Compound 48, [2-(3-(((R)-3-(isoquinolin-5-ylamino)pyrrolidin-1-yl)methyl)phenoxy)ethanol], and Compound 49, [2-(5-(((R)-3-(isoquinolin-5-ylamino)pyrrolidin-1-yl)methyl)-2-methylphenoxy)] ethanol, are shown below.

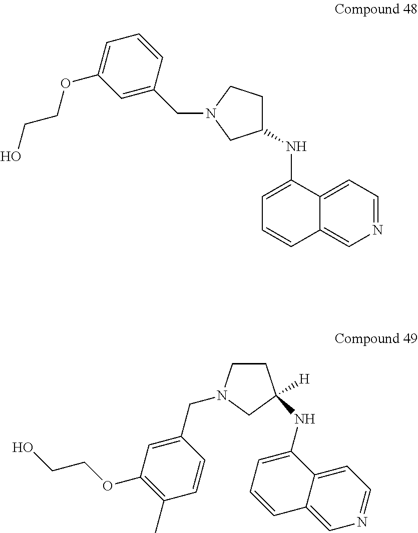

TABLE 1
ROCK1 and ROCK2 IC₅₀ results

| Compound # | ROCK1 IC₅₀ (μM) | ROCK2 IC₅₀ (μM) |
|---|---|---|
| 14 | 2.46 | 0.717 |
| 15 | | |
| 16 | | |
| 17 | 1.22 | 0.369 |
| 18 | 3.82 | 1.49 |
| 19 | 3.06 | 1.05 |
| 20 | 4.81 | 1.77 |
| 21 | 1.91 | 0.512 |
| 22 | | |
| 23 | | |
| 24 | | 2.75 |
| 25 | | 2.30 |
| 26 | 6.06 | 0.621 |
| 27 | 3.44 | 0.251 |
| 28 | 1.03 | 0.109 |
| 29 | 5.16 | 0.987 |
| 30 | 5.39 | 0.451 |
| 31 | 5.65 | 1.23 |
| 32 | 7.08 | 3.02 |
| 33 | 1.01 | 0.155 |
| 34 | 1.28 | 0.102 |
| 35 | | |
| 36 | 0.545 | 0.246 |
| 37 | | 0.591 |
| 38 | | 6.19 |
| 39 | | 3.71 |
| 40 | | 6.59 |
| 41 | | 0.087 |
| 42 | 2.42 | 0.341 |
| 43 | 25.4 | 4.00 |
| 44 | 116.9 | 6.19 |
| 45 | 45.5 | 6.18 |
| 46 | | |
| 48 | 0.019 | 0.0067 |
| 49 | 0.0041 | 0.0022 |

### Example48

### Ocular Comfort

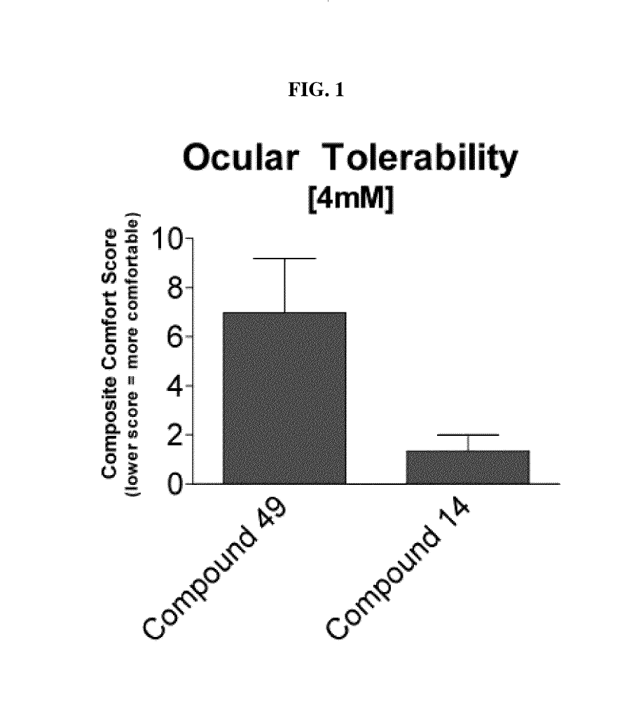

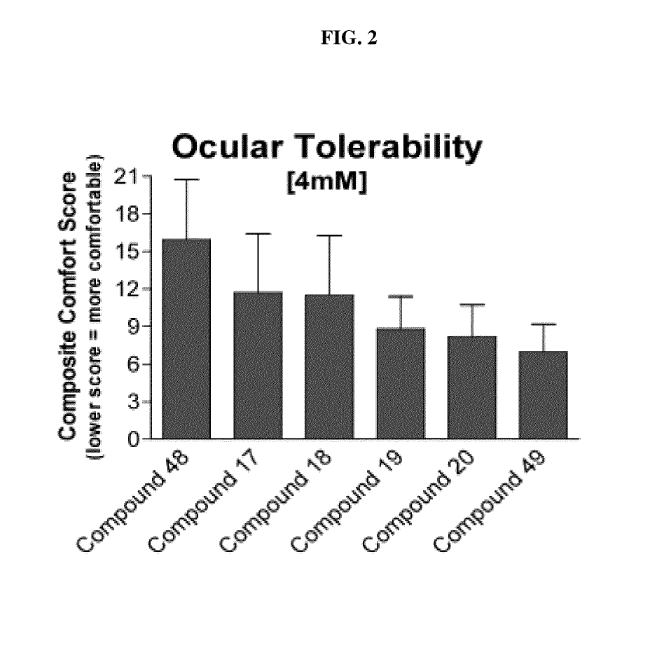

The desired compound at a concentration of 4 mM in a formulation of 10 mM phosphate, 1% polysorbate 80, 0.85% NaCl, 0.02% BAC, 0.2% EDTA pH 7.0 was administered as two 30 82 l drops to the right eye of each rabbit within a dosing group. The rabbits were evaluated for 15 minutes after ocular instillation and their changes in behavior were recorded. A composite score for each rabbit within each treatment group was created based upon the number of times they demonstrated a unilateral blink, bilateral blink, front paw wipe of the face, scratch and head shake. The higher the score, the more discomfort an animal senses. A mean ±SE was generated for each group and depicted in FIGS. 1 and 2.

FIG. 1 shows that corresponding ester prodrugs (Compound 14) elicit a reduced level of discomfort compared to the parent compound [2-(5-(((R)-3-(isoquinolin-5-ylamino)pyrrolidin-1-yl)methyl)-2-methylphenoxy)]ethanol (Compound 49).

FIG. 2 shows that corresponding ester prodrugs (Compounds 17-20) elicit a reduced level of discomfort compared to the parent compound [2-(3-(((R)-3-(isoquinolin-5-ylamino)pyrrolidin-1-yl)methyl)phenoxy)ethanol] (Compound 48). Compound 49 was included in the figure only to show relevance to FIG. 1.

### Example 49

### Ocular Pharmacokinetic Assay

Intraocular fluid (aqueous humor) was collected from New Zealand White rabbits to determine corneal and anterior chamber pharmacokinetics of formulations containing Compounds 17, 18, 19, 20, 21, and 48. Compounds 17, 18, 19, 20, 21 are prodrugs of Compound 48. Each animal was dosed bilaterally with 1×30 μl of 1 mM of each test compound (in 10 mM phosphate, 0.8% polysorbate 80, 0.85% NaCl, 0.01% BAC, 0.1% EDTA at pH 7.3). During instillation, the upper and lower eyelids were immobilized and the compound was administered to the superior aspect of the globe allowing it to flow across the ocular surface. Following instillation, blinking was prevented for 30 seconds. Aqueous humor was collected after 1 hour following topical instillation using a 30-gauge needle inserted proximal to the corneal scleral limbus. Subsequently 30 μl of aqueous humor was aspirated using a 300 μl syringe. Aqueous humor samples were assayed for the concentration of the test compound using an LC/MS/MS assay system. All experiments were conducted in accordance with the ARVO Statement for the Use of Animals in Ophthalmic and Vision Research and in compliance with National Institutes of Health. The results of observed aqueous humor concentrations of the test compounds at 1 hour post-instillation in the animal eyes are described in Table 2.

TABLE 2
Concentrations of the parent compound (Compound 48) in the
aqueous humor following dosing of 5 prodrugs (and the base
compound) at a concentration of 1 mM (pH = 7.3) in a 1 × 30 μL
administration to the ocular surface (time point of 1 hour);

| | | Concentration of | |
|---|---|---|---|
| Prodrug/ | | Compound 48 | |
| (base) | | (nM) | SD |
| Compound 17 | n = 4 | 97.81 | 32.65 |
| Compound 18 | n = 4 | 70.57 | 32.02 |
| Compound 19 | n = 4 | 78.55 | 27.55 |
| Compound 20 | n = 4 | 79.03 | 23.16 |
| Compound 21 | n = 4 | 119.75 | 58.04 |
| (Compound 48) | n = 4 | 62.80 | 9.55 |

The results show that prodrug Compounds 17-21, when dosed topically, were able to penetrate the eye and achieved concentrations in the aqueous humor higher than that provided by base Compound 48.

### Example 50

### Ocular Surface and Aqueous Humor Bioavailability

Dose Formulation and Administration. Compounds 14 (prodrug) and 49 (base compound) were formulated at 0.04% w/v (the equivalent millimolar concentration is 1 mM) in 10 mM phosphate, 0.8% polysorbate 80, 0.85% NaCl, 0.01% BAC, 0.1% EDTA at pH 7.3. Each compound was administered as a 30 μl drop to both eyes of each animal within a dosing group and the ocular and systemic exposure was examined as described in Example 49.

Study sampling. 40 μL of saline was applied to the eyes at 0.083, 1, 2, and 4 hours after administration of each compound and the lavage fluids were collected as samples. Aqueous humor and ocular surface samples were obtained from 2 animals (4 eyes) per dosing group at 0.083, 1, 2, and 4 hours post dosing, by the methods described in Example 49. Ocular surface relates to the surface of the cornea and conjunctiva. Ocular surface residence time is the average time that a compound resides on the ocular surface.

Table 3 shows the ocular surface and aqueous humor concentration of Compounds 14 and 49 over time after administration of Compound 14. Table 4 shows the ocular surface and aqueous humor concentration of Compound 49 over time after administration of Compound 49.

TABLE 3
Aqueous humor and ocular surface concentration
of a prodrug and its base compound
Compound 14 dosed at a concentration of
1 mM (pH = 7.3) in a 1 × 30 uL administration to 4 eyes

| | | Aqueous Humor | | Ocular Surface | |
|---|---|---|---|---|---|
| time (h) | | [14] | [49] | [14] | [49] |
| 0.83 | Mean | 0 | 109.4 | 24290 | 74034 |
| | SE | 0 | 101.7 | 11483 | 6893 |
| 1 | Mean | 0 | 152.3 | 3081.4 | 791.6 |
| | SE | 0 | 24.4 | 542.7 | 231.8 |
| 2 | Mean | 0 | 13.4 | 245.5 | 703.57 |
| | SE | 0 | 2.2 | 23 | 226.5 |
| 4 | Mean | 0 | 6 | 0 | 39.6 |
| | SE | 0 | 0.5 | 0 | 18 |

Numbers shown are concentrations of prodrug (Compound 14) and parent compound (Compound 49) in nM at each time point

TABLE 4
Aqueous humor and ocular surface concentrations
of Compound 49
Compound 49 dosed at a concentration of 1 mM
(pH = 7.3) in a 1 × 30 uL administration to 4 eyes

| time (h) | | Aqueous Humor | Ocular Surface |
|---|---|---|---|
| 0.83 | Mean | 11.9 | 146761 |
| | SE | 7.6 | 75889 |
| 1 | Mean | 93.9 | 846.5 |
| | SE | 47.3 | 196 |
| 2 | Mean | 24.0 | 892.7 |
| | SE | 4.2 | 660.1 |
| 4 | Mean | 3.4 | 65.7 |
| | SE | 0.4 | 21.1 |

Numbers shown are concentrations of Compound 49 in nM at each time point

What is claimed:

1. A compound of Formula I, or its pharmaceutically acceptable salt, tautomers thereof, 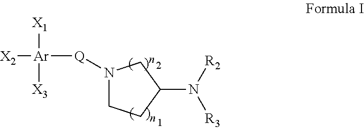
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 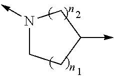
is optionally substituted by alkyl, halo, oxo, OR₆, NR₆R₇, or SR₆;
R₂ is selected from the following heteroaryl systems, optionally substituted: 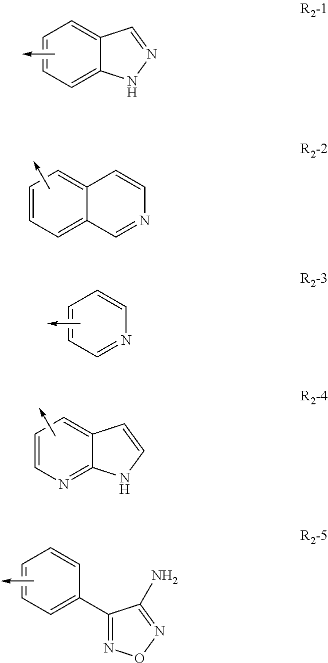
R₃—R₇ are independently H, alkyl, alkenyl, alkynyl, cycloalkyl, cycloalkenyl, cycloalkylalkyl, cycloalkylalkenyl, or cycloalkylalkynyl, optionally substituted;
Ar is a monocyclic or bicyclic aryl or heteroaryl ring;
X₁ is -J₁C(O)R₁₀ or -J₁(CR₈R₉)n₄J₂C(O)R₁₀, with n₄=1-6 and J₁ and J₂ are independently O, NR₁₂, or absent;
X₂ and X₃ are independently H, halogen, OR₁₂, NR₁₂R₁₃, SR₁₂, SOR₁₂, SO₂R₁₂, SO₂NR₁₂R₁₃, OCF3, saturated or unsaturated heterocycle, heteroaryl, aryl, alkyl, alkenyl, or alkynyl;
R₈, R₉ are independently H, halogen, alkyl (n=1-3), alkyloxy, alkylthio, or OR₁₁;
R₁₀ is alkyl, alkenyl, heterocycle, aryl, heteroaryl, aralkyl, cycloalkyl, each optionally substituted; or R₁₀ is OR₁₂ or NR₁₂R₁₃;
R₁₁=H or alkyl (n=1-3); and
R₁₂ and R₁₃ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle, optionally substituted;
provided that when Q=CH₂; n₁=n₂=1; R₂=R₂−2; R₃=H; Ar=phenyl; X₂ and X₃=H; X₁=OCH₂CH₂OC(O)R₁₂, then R₁₂ is not phenyl.

2. The compound according to claim 1, wherein R₂ is R₂−1 or R₂−2.

3. The compound according to claim 1, wherein Q is (CR₄R₅)ₙ₃, n₁ is 1 or 2; n₂ is 1; n₃ is 1 or 2; and R₃—R₇ are H.

4. The compound according to claim 1, wherein X₁ is -J₁C(O)R₁₀.

5. The compound according to claim 1, wherein X₁ is J₁(CR₈R₉)n₄J₂C(O)R₁₀.

6. The compound according to claim 1, wherein J₂ is O or NR₁₂, J₁ is absent or 0.

7. The compound according to claim 1, wherein X₂ and X₃ are H.

8. The compound according to claim 1, wherein said Formula I compound is Compound 14, 2-(5-(((R)-3-(isoquinolin-5-ylamino)pyrrolidin-1-yl)methyl)-2-methylphenoxy)ethyl benzoate; Compound 15, (R)-tert-butyl 2-(5-((3-(isoquinolin-5-ylamino)pyrrolidin-1-yl)methyl)-2-methylphenoxy)acetate; Compound 16, 2-(3-((3-(isoquinolin-5-ylamino)pyrrolidin-1-yl)methyl)phenoxy)ethyl benzoate; Compound 17, 2-(3-(((R)-3-(isoquinolin-5-ylamino)pyrrolidin-1-yl)methyl)phenoxy)ethyl ethyl carbonate; Compound 18, 2-(3-((((R)-3-(isoquinolin-5-ylamino)pyrrolidin-1-yl)methyl)phenoxy)ethyl 3-methylbutanoate); Compound 19, 2-(3-(((R)-3-(isoquinolin-5-ylamino)pyrrolidin-1-yl)methyl)phenoxy)ethyl 1-methylcyclopropanecarboxylate; Compound 20, 2-(3-(((R)-3-(isoquinolin-5-ylamino)pyrrolidin-1-yl)methyl)phenoxy)ethyl pivalate; or Compound 21, 2-(3-(((R)-3-(isoquinolin-5-ylamino)pyrrolidin-1-yl)methyl)phenoxy)ethyl nicotinate.

9. The compound according to claim 1, wherein said Formula I compound is Compound 22, 2-(3-(((R)-3-(isoquinolin-5-ylamino)pyrrolidin-1-yl)methyl)phenoxy)ethyl benzoate; Compound 24, N-(4-((3-1H-indazol-5-ylamino)pyrrolidin-1-yl)methyl)phenyl)acetamide; Compound 25, N-(4-((3-(isoquinolin-5-ylamino)pyrrolidin-1-yl)methyl)phenyl)acetamide; Compound 26, 2-(5-(((R)-3-(1H-indazol-5-ylamino)piperidin-1-yl)methyl)-2-methylphenoxy)ethyl benzoate; Compound 27, tert-Butyl 2-(3-(((S)-3-(1H-indazol-5-ylamino)piperidin-1-yl)methyl)phenoxy) acetate; Compound 28; Ethyl 2-(3-(((S)-3-(1H-indazol-5-ylamino)piperidin-1-yl)methyl)phenoxy)acetate; or Compound 29, N-(2-(3-(((R)-3-(1H-indazol-5-ylamino)piperidin-1-yl)methyl)phenoxy)ethyl) acetamide.

10. The compound according to claim 1, wherein said Formula I compound is Compound 30, N-(2-(3-(((S)-3-(1H-indazol-5-ylamino)piperidin-1-yl)methyl)phenoxy)ethyl) acetamide; Compound 31, 2-(3-(((S)-3-(1H-indazol-5-ylamino)piperidin-1-yl)methyl)phenoxy)ethyl benzoate, Compound 32, 2-(3-((R)-3-(1H-indazol-5-ylamino)piperidin-1-yl)methyl)phenoxy)ethyl benzoate; Compound 33, 2-(3-(((R)-3-(1H-indazol-5-ylamino)piperidin-1-yl)methyl)phenoxy)-N-(pyridin-3-yl)acetamide; Compound 34, 2-(3-(((R)-3-(1H-indazol-5-ylamino)piperidin-1-yl)methyl)phenoxy)-1-morpholinoethanone; Compound 35, 2-(3-(((R)-3-(1H-indazol-5-ylamino)piperidin-1-yl)methyl)phenoxy)-1-(4-methylpiperazin-1-y1)ethanone; Compound 36, Ethyl 2-(3-(((R)-3-(1H-indazol-4-ylamino)piperidin-1-yl)methyl)phenoxy)acetate; or Compound 37, N-(2-(3-((3-1H-indazol-5-ylamino)piperidin-1-yl)methyl)phenoxy)ethyl)acetamide.

11. The compound according to claim 1, wherein said Formula I compound is Compound 38, N-(4-((3-(1H-indazol-5-ylamino)piperidin-1-yl)methyl)phenyl)acetamide; Compound 39, N-(4-((3-isoquinolin-5-ylamino)piperidin-1-yl)methyl)phenyl)acetamide; Compound 40, tert-Butyl (3-((3-(1H-indazol-5-ylamino)piperidin-1-yl)methyl)phenyl)methyl carbamate; Compound 41, Ethyl 2-(3-((3-(1H-indazol-5-ylamino)piperidin-1-yl)methyl)phenoxy)acetate; Compound 42, N-((3-(((R)-3-(1H-indazol-5-ylamino)piperidin-1-yl)methyl)phenyl)methyl) acetamide; Compound 43, tert-Butyl (4-(((S)-3-(1H-indazol-5-ylamino)piperidin-1-yl)methyl)phenyl) methylcarbamate; Compound 44, Ethyl 4-(((R)-3-(1H-indazol-5-ylamino)piperidin-1-yl)methyl)benzoate; Compound 45, Ethyl 4-(((S)-3-(1H-indazol-5-ylamino)piperidin-1-yl)methyl)benzoate; or Compound 46, 2-(3-((3-(isoquinolin-5-ylamino)pyrrolidin-1-yl)methyl)phenoxy)ethyl acetate.

12. A pharmaceutical composition comprising the compound according claim 1 and a pharmaceutically acceptably carrier.

13. A method of treating an ophthalmic disease selected from the group consisting of glaucoma, allergic conjunctivitis, macular edema, macular degeneration, and blepharitis; comprising the steps of:
identifying a subject suffering from glaucoma, allergic conjunctivitis, macular edema, macular degeneration, or blepharitis; and
administering to the subject an effective amount of the compound according to claim 1.

14. The method according to claim 13, wherein said administering is topical administering.

15. A method of treating intraocular pressure; comprising the steps of:
identifying a subject suffering from glaucoma, allergic conjunctivitis, macular edema, macular degeneration, or blepharitis; and
administering to the subject an effective amount of the compound according to claim 1.

16. The method according to claim 15, wherein said administering is topical administering.
